(19) United States
(12) Patent Application Publication
(10) Pub. No.: US 2024/0383892 A1
(43) Pub. Date: Nov. 21, 2024

(54) PARTICLES OF IMIDAZO[4,5-b]PYRIDINE COMPOUND, PHARMACEUTICAL COMPOSITIONS, AND THEIR USE IN TREATING MEDICAL CONDITIONS

(71) Applicant: Centrexion Therapeutics Corporation, Boston, MA (US)

(72) Inventors: Jonathan Charles WALKER, North Andover, MA (US); Joanna BIS, Morrisville, NC (US); Wei MA, Cary, NC (US)

(21) Appl. No.: 18/667,683

(22) Filed: May 17, 2024

Related U.S. Application Data
(60) Provisional application No. 63/467,067, filed on May 17, 2023.

(51) Int. Cl. C07D 471/04; A61K 9/107; A61K 9/16; A61K 31/437; A61K 47/26; A61K 47/28; A61K 47/32; A61P 29/00

(52) U.S. Cl. CPC C07D 471/04; A61K 9/107; A61K 9/16; A61K 31/437; A61K 47/26; A61K 47/28; A61K 47/32; A61P 29/00

(57) ABSTRACT

The invention provides particles of an imidazo[4,5-b]pyridine compound having a preferred particle size range, pharmaceutical compositions, methods of inhibiting tropomyosin-related kinase and/or c-FMS, methods of treating medical diseases and conditions, such as pain, and methods for preparing such particles.

### CROSS REFERENCE TO RELATED APPLICATIONS

This application claims the benefit of and priority to U.S. Provisional Patent Application Ser. No. 63/467,067, filed May 17, 2023, the contents of which are hereby incorporated by reference.

### FIELD OF THE INVENTION

The invention provides particles of an imidazo[4,5-b]pyridine compound having a preferred particle size range, pharmaceutical compositions, methods of inhibiting tropomyosin-related kinase and/or c-FMS, methods of treating medical diseases and conditions, such as pain, and methods for preparing such particles.

### BACKGROUND

Pain can function as a protective mechanism that allows healthy human beings and animals to avoid tissue damage and/or prevent further damage to injured tissue. However, there are many instances in which pain persists beyond its usefulness. Such unnecessary suffering from pain can impair a person's physical mobility, mental performance, ability to sleep normally, to engage in occupations, and even contribute to depression. One type of pain that affects a substantial number of patients is osteoarthritis pain. Osteoarthritis pain can be debilitating. For example, patients suffering from knee osteoarthritis pain are often impaired in their ability to perform simple daily tasks such as walking or climbing stairs. The pain may be felt even while sitting in a chair or lying in bed and may interfere with ability to sleep. Long duration relief from knee osteoarthritis pain would provide a substantial benefit to patients suffering from knee osteoarthritis pain.

Compounds that inhibit tropomyosin-related kinase have been reported for use in treating pain, such as osteoarthritis pain. Tropomyosin-related kinases are high affinity receptors activated by soluble growth factors called neutrophins. Activation of a tropomyosin-related kinase leads to the activation of downstream kinases that are implicated in cell signaling, including cell proliferation, survival, angiogenesis and metastasis. International patent application publications WO 2015/089139, WO 2016/100677, and WO 2023/086564 describe certain compounds that inhibit tropomyosin-related kinase. Additional compositions for inhibiting tropomyosin-related kinase and having superior properties are desirable.

The present invention addresses this need and provides other related advantages.

### SUMMARY

The invention provides particles of an imidazo[4,5-b]pyridine compound having a preferred particle size range, pharmaceutical compositions, methods of inhibiting tropomyosin-related kinase and/or c-FMS, methods of treating medical diseases and conditions, such as pain, and methods for preparing such particles. For example, one aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm. Another aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D90 particle size in the range of from 70 μm to 150 μm. In a preferred embodiment, the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline form, such as the crystalline form characterized according to an X-ray powder diffraction pattern comprising peaks at the following diffraction angles (2θ): 14.9 0.2, 20.2±0.2, 20.7±0.2, 21.4±0.2, 25.1±0.2, 28.0±0.2, and 30.0±0.2. Superior performance properties were unexpectedly discovered for certain size particles of the crystalline compound 3-(3-methoxy-4-((4-methoxybenzyl)oxy) benzyl)-6-(1-methyl-1H-pyrazol-4-yl)-3H-imidazo[4,5-b]pyridin-2-amine monohydrate characterized according to an X-ray powder diffraction pattern comprising peaks at diffraction angles (2θ) 14.9±0.2, 20.2±0.2, 20.7±0.2, 21.4±0.2, 25.1±0.2, 28.0±0.2, and 30.0±0.2. For example, significantly less of the adverse side effect, inflammation, was observed upon administration to the subject certain size particles of the crystalline compound 3-(3-methoxy-4-((4-methoxybenzyl)oxy)benzyl)-6-(1-methyl-1H-pyrazol-4-yl)-3H-imidazo[4,5-b]pyridin-2-amine monohydrate characterized according to an X-ray powder diffraction pattern comprising peaks at diffraction angles (2θ) 14.9±0.2, 20.2±0.2, 20.7±0.2, 21.4±0.2, 25.1±0.2, 28.0±0.2, and 30.0±0.2. The particles may be used in pharmaceutical compositions and therapeutic methods described herein. Various aspects and embodiments are described in more detail below.

Another aspect of the invention provides a method of treating a disease or condition selected from the group consisting of an inflammatory disease, autoimmune disease, pain, osteoarthritis, defect of bone metabolism, and cancer. The method comprises administering a therapeutically effective amount of particles described herein to a subject in need thereof to treat the disease or condition. In certain embodiments, the disease or condition is pain, such as pain due to osteoarthritis. In certain embodiments, the disease or condition is osteoarthritis.

Another aspect of the invention provides a method of inhibiting the activity of a tropomyosin-related kinase. The method comprises contacting a tropomyosin-related kinase with an effective amount of particles described herein to inhibit the activity of said tropomyosin-related kinase. In certain embodiments, the tropomyosin-related kinase is tropomyosin-related kinase A. In certain embodiments, the tropomyosin-related kinase is tropomyosin-related kinase B. In certain embodiments, the tropomyosin-related kinase is tropomyosin-related kinase C.

Another aspect of the invention provides a method of inhibiting the activity of a cellular receptor for colony stimulating factor-1. The method comprises contacting said cellular receptor for colony stimulating factor-1 with an effective amount of particles described herein to inhibit the activity of said cellular receptor for colony stimulating factor-1.

Another aspect of the invention provides a method of preparing particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm, the method comprises the steps of:
a. admixing (i) a first solution having a temperature of about 50° C. and containing acetone, water, and 3-(3-methoxy-4-((4-methoxybenzyl)oxy)benzyl)-6-(1-methyl-1H-pyrazol-4-yl)-3H-imidazo[4,5-b]pyridin-2-amine and (ii) an aliquot of 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form to provide a first mixture;
b. maintaining the first mixture at a temperature in the range of from 42° C. to 48° C. for a duration of about 1 hour, then cooling the first mixture to a temperature in the range of from 37° C. to 42° C. and maintaining the first mixture at a temperature in the range of from 37° C. to 42° C. for about 1 hour, then cooling the first mixture to a temperature in the range of from 2° C. to 8° C. and maintaining the first mixture at a temperature in the range of from 2° C. to 8° C. for about 1 hour, to produce a second mixture;
c. heating the second mixture to a temperature in the range of from 48° C. to 53° C., maintaining the second mixture at a temperature in the range of from 48° C. to 53° C. for about 3 hours, then cooling the second mixture to a temperature in the range of from 43° C. to 47° C. and maintaining the second mixture at a temperature in the range of from 43° C. to 47° C. for about 1 hour, then cooling the second mixture to a temperature in the range of from 37° C. to 42° C. and maintaining the second mixture at a temperature in the range of from 37° C. to 42° C. for about 1 hour, then cooling the second mixture to a temperature in the range of from 2° C. to 8° C. and maintaining the second mixture at a temperature in the range of from 2° C. to 8° C. for about 1 hour, to produce a third mixture;
d. heating the third mixture to a temperature in the range of from 48° C. to 53° C., maintaining the third mixture at a temperature in the range of from 48° C. to 53° C. for about 3 hours, then cooling the third mixture to a temperature in the range of from 43° C. to 47° C. and maintaining the third mixture at a temperature in the range of from 43° C. to 47° C. for about 1 hour, then cooling the third mixture to a temperature in the range of from 37° C. to 42° C. and maintaining the third mixture at a temperature in the range of from 37° C. to 42° C. for about 1 hour, then cooling the third mixture to a temperature in the range of from 2° C. to 8° C. and maintaining the third mixture at a temperature in the range of from 2° C. to 8° C. for about 1 hour, to produce a crystallization mixture; and
e. isolating said particles from the crystallization mixture.

Further embodiments are described herein below.

### BRIEF DESCRIPTION OF THE DRAWINGS

FIG. 1 depicts an X-ray powder diffractogram of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3, as further described in Example 3.

FIG. 2 depicts a differential scanning calorimetry curve of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3, as further described in Example 3.

FIG. 3 depicts a DVS isotherm plot of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3, as further described in Example 3.

FIG. 4 depicts a thermogravimetric analysis/mass spectrometry (TGA-MS) profile of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3, as further described in Example 3.

FIG. 5 depicts an X-ray powder diffractogram of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1.

FIG. 6 depicts an X-ray powder diffractogram of taken on particles of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3, as further described in Example 5. The lower XRPD diffractogram in FIG. 6 is the X-ray powder diffractogram taken on particles of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 prepared in Example 5. The upper XRPD diffractogram in FIG. 6 was taken on the micronized 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 used as starting material in Example 5 to make the particles.

FIG. 7 illustrates a polarized light microscopy image that was obtained on the particles of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 prepared in Example 5, as further described in Example 5.

FIG. 8 depicts graphs showing (i) a differential scanning calorimetry curve obtained from analyzing particles of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 prepared in Example 5 and (ii) a thermogravimetric analysis profile of the particles of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 prepared in Example 5, as further described in Example 5.

FIG. 9 is a graph showing results of rat knee swelling resulting from administration of different formulations containing 3-(3-methoxy-4-((4-methoxybenzyl) oxy)benzyl)-6-(1-methyl-1H-pyrazol-4-yl)-3H-imidazo[4,5-b]pyridin-2-amine monohydrate, as further described in Example 6.

FIG. 10 is a graph showing results of rat knee swelling resulting from administration of different formulations containing 3-(3-methoxy-4-((4-methoxybenzyl) oxy)benzyl)-6-(1-methyl-1H-pyrazol-4-yl)-3H-imidazo[4,5-b]pyridin-2-amine monohydrate, as further described in Example 7.

FIG. 11 is a graph showing results demonstrating ability of different formulations containing 3-(3-methoxy-4-((4-methoxybenzyl) oxy)benzyl)-6-(1-methyl-1H-pyrazol-4-yl)-3H-imidazo[4,5-b]pyridin-2-amine monohydrate to prevent rat knee pain, as further described in Example 8.

FIG. 12 is a graph showing rat paw inflammation resulting from administration of different formulations containing 3-(3-methoxy-4-((4-methoxybenzyl) oxy)benzyl)-6-(1-methyl-1H-pyrazol-4-yl)-3H-imidazo[4,5-b]pyridin-2-amine monohydrate, as further described in Example 9.

### DETAILED DESCRIPTION

The invention provides particles of an imidazo[4,5-b]pyridine compound having a preferred particle size range, pharmaceutical compositions, methods of inhibiting tropomyosin-related kinase and/or c-FMS, methods of treating medical diseases and conditions, such as pain, and methods for preparing such particles. For example, one aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm. Another aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D90 particle size in the range of from 70 μm to 150 μm. In a preferred embodiment, the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline form, such as the crystalline form characterized according to an X-ray powder diffraction pattern comprising peaks at the following diffraction angles (2θ): 14.9 0.2, 20.2±0.2, 20.7±0.2, 21.4±0.2, 25.1±0.2, 28.0±0.2, and 30.0±0.2. Superior performance properties were unexpectedly discovered for certain size particles of the crystalline compound 3-(3-methoxy-4-((4-methoxybenzyl)oxy) benzyl)-6-(1-methyl-1H-pyrazol-4-yl)-3H-imidazo[4,5-b]pyridin-2-amine monohydrate characterized according to an X-ray powder diffraction pattern comprising peaks at diffraction angles (2θ) 14.9±0.2, 20.2±0.2, 20.7±0.2, 21.4±0.2, 25.1±0.2, 28.0±0.2, and 30.0±0.2. For example, significantly less of the adverse side effect, inflammation, was observed upon administration to the subject certain size particles of the crystalline compound 3-(3-methoxy-4-((4-methoxybenzyl)oxy)benzyl)-6-(1-methyl-1H-pyrazol-4-yl)-3H-imidazo[4,5-b]pyridin-2-amine monohydrate characterized according to an X-ray powder diffraction pattern comprising peaks at diffraction angles (2θ) 14.9±0.2, 20.2±0.2, 20.7±0.2, 21.4±0.2, 25.1±0.2, 28.0±0.2, and 30.0±0.2. The particles may be used in pharmaceutical compositions and therapeutic methods described herein. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates.

The terms “a” and “an” as used herein mean “one or more” and include the plural unless the context is inappropriate.

As used herein, the terms “subject” and “patient” are used interchangeably and refer to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

Unless specified otherwise, the term “about” refers to within ±10% of the stated value. The invention encompasses embodiments where the value is within ±9%, ±8%, ±7%, ±6%, ±5%, ±4%, ±3%, ±2%, or ±1% of the stated value.

The chemical name “3-(3-methoxy-4-((4-methoxybenzyl)oxy)benzyl)-6-(1-methyl-1H-pyrazol-4-yl)-3H-imidazo[4,5-b]pyridin-2-amine” refers to the compound having the following formula:

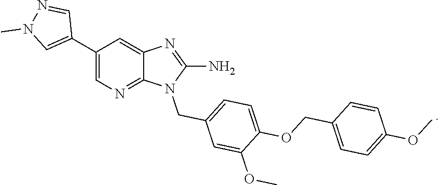

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄+(wherein W is a C₁₋₄ alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

The invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Schemes and Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Particles of 3-(3-Methoxy-4-((4-methoxybenzyl)oxy)benzyl)-6-(1-methyl-1H-pyrazol-4-yl)-3H-imidazo[4,5-b]pyridin-2-amine

The invention provides particles of the compound 3-(3-methoxy-4-((4-methoxy-benzyl)oxy)benzyl)-6-(1-methyl-1H-pyrazol-4-yl)-3H-imidazo[4,5-b]pyridin-2-amine. Preferably, the particles have a D50 particle size in the range of from 40 μm to 75 μm. Exemplary features and further embodiments are described herein below.

Accordingly, one aspect the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine having a D50 particle size in the range of from 40 μm to 75 μm. The particles may be further characterized as set forth below.

Another aspect the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm. The particles may be further characterized as set forth below.

Another aspect the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm, wherein the 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline form characterized according to an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2. The particles may be further characterized as set forth below.

Another aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine having a D90 particle size in the range of from 70 μm to 150 μm. The particles may be further characterized as set forth below.

Another aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D90 particle size in the range of from 70 μm to 150 μm. The particles may be further characterized as set forth below.

Another aspect of the invention provides particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D90 particle size in the range of from 70 μm to 150 μm, wherein the 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline form characterized according to an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2. The particles may be further characterized as set forth below.

The particles may be further characterized according to, for example, particle size, physical form of the 3-(3-methoxy-4-((4-methoxybenzyl)oxy)benzyl)-6-(1-methyl-1H-pyrazol-4-yl)-3H-imidazo[4,5-b]pyridin-2-amine, amount of compound in the particles that is in the form of plate-like crystals, amount of compound in the particles that is in the form needle-like crystals, and other features.

### Particle Size

The particles may be further characterized according to particle size. Particle size may be characterized according to, for example, D10, D50, and/or D90 values. For example, in certain embodiments, the particles have a D10 particle size in the range of from 12 μm to 26 μm. In certain embodiments, the particles have a D10 particle size in the range of from 14 μm to 24 μm. In certain embodiments, the particles have a D10 particle size in the range of from 16 μm to 22 μm. In certain embodiments, the particles have a D10 particle size in the range of from 17 μm to 21 μm. In certain embodiments, the particles have a D10 particle size in the range of from 18 μm to 19 μm. In certain embodiments, the particles have a D10 particle size of about 12 μm, 13 μm, 14 μm, 15 μm, 16 μm, 17 μm, 18 μm, 19 μm, 20 μm, 21 μm, 22 μm, 23 μm, 24 μm, 25 μm, or 26 μm. In certain embodiments, the particles have a D10 particle size of about 17 μm. In certain embodiments, the particles have a D10 particle size of about 18 μm. In certain embodiments, the particles have a D10 particle size of about 19 μm. In certain embodiments, the particles have a D10 particle size of about 20 μm. In certain embodiments, the particles have a D10 particle size of about 21 μm. In certain embodiments, the particles have a D10 particle size of 17 μm±2 μm. In certain embodiments, the particles have a D10 particle size of 18 μm±2 μm. In certain embodiments, the particles have a D10 particle size of 19 μm±2 μm. In certain embodiments, the particles have a D10 particle size of 20 μm±2 μm. In certain embodiments, the particles have a D10 particle size of 21 μm±2 μm. In certain embodiments, the particles have a D10 particle size of 17 μm±1 μm. In certain embodiments, the particles have a D10 particle size of 18 μm±1 μm. In certain embodiments, the particles have a D10 particle size of 19 μm±1 μm. In certain embodiments, the particles have a D10 particle size of 20 μm±1 μm. In certain embodiments, the particles have a D10 particle size of 21 μm±1 μm.

In certain embodiments, the particles have a D10 particle size in the range of from 5 μm to 35 μm. In certain embodiments, the particles have a D10 particle size in the range of from 5 μm to 30 μm. In certain embodiments, the particles have a D10 particle size in the range of from 5 μm to 10 μm. In certain embodiments, the particles have a D10 particle size in the range of from 7 μm to 9 μm. In certain embodiments, the particles have a D10 particle size in the range of from 6 μm to 8 μm. In certain embodiments, the particles have a D10 particle size in the range of from 6 μm to 9 μm. In certain embodiments, the particles have a D10 particle size in the range of from 20 μm to 30 μm. In certain embodiments, the particles have a D10 particle size in the range of from 23 μm to 29 μm. In certain embodiments, the particles have a D10 particle size in the range of from 24 μm to 28 μm. In certain embodiments, the particles have a D10 particle size in the range of from 12 μm to 18 μm. In certain embodiments, the particles have a D10 particle size in the range of from 10 μm to 15 μm. In certain embodiments, the particles have a D10 particle size in the range of from 11 μm to 14 μm. In certain embodiments, the particles have a D10 particle size in the range of from 12 μm to 13 μm. In certain embodiments, the particles have a D10 particle size in the range of from 12 μm to 14 μm. In certain embodiments, the particles have a D10 particle size in the range of from 14 μm to 20 μm. In certain embodiments, the particles have a D10 particle size in the range of from 15 μm to 19 μm. In certain embodiments, the particles have a D10 particle size in the range of from 16 μm to 18 μm. In certain embodiments, the particles have a D10 particle size of about 6 μm, 7 μm, 8 μm, 9 μm, 10 μm, 11 μm, or 12 μm. In certain embodiments, the particles have a D10 particle size of 10 μm±2 μm, 11 μm±2 μm, or 12 μm 2 μm. In certain embodiments, the particles have a D10 particle size of 6 μm±1 μm, 7 μm 1 μm, 8 μm±1 μm, 9 μm±1 μm, 10 μm±1 μm, 11 μm±1 μm, or 12 μm±1 μm. In certain embodiments, the particles have a D10 particle size of about 27 μm, 28 μm, 29 μm, or 30 μm. In certain embodiments, the particles have a D10 particle size of about 27 μm±2 μm, 28 μm±2 μm, 29 μm±2 μm, or 30 μm±2 μm.

In certain embodiments, the particles have a D50 particle size in the range of from 40 μm to 75 μm. In certain embodiments, the particles have a D50 particle size in the range of from 47 μm to 67 μm. In certain embodiments, the particles have a D50 particle size in the range of from 52 μm to 62 μm. In certain embodiments, the particles have a D50 particle size in the range of from 55 μm to 59 μm. In certain embodiments, the particles have a D50 particle size in the range of from 56 μm to 57 μm. In certain embodiments, the particles have a D50 particle size of about 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, or 75 μm. In certain embodiments, the particles have a D50 particle size of about 55 μm. In certain embodiments, the particles have a D50 particle size of about 56 μm. In certain embodiments, the particles have a D50 particle size of about 57 μm. In certain embodiments, the particles have a D50 particle size of about 58 μm. In certain embodiments, the particles have a D50 particle size of about 59 μm. In certain embodiments, the particles have a D50 particle size of 55 μm±2 μm. In certain embodiments, the particles have a D50 particle size of 56 μm±2 μm. In certain embodiments, the particles have a D50 particle size of 57 μm±2 μm. In certain embodiments, the particles have a D50 particle size of 58 μm±2 μm. In certain embodiments, the particles have a D50 particle size of 59 μm±2 μm. In certain embodiments, the particles have a D50 particle size of 55 μm±1 μm. In certain embodiments, the particles have a D50 particle size of 56 μm±1 μm. In certain embodiments, the particles have a D50 particle size of 57 μm±1 μm. In certain embodiments, the particles have a D50 particle size of 58 μm±1 μm. In certain embodiments, the particles have a D50 particle size of 59 μm±1 μm.

In certain embodiments, the particles have a D50 particle size in the range of from 30 μm to 65 μm. In certain embodiments, the particles have a D50 particle size in the range of from 30 μm to 60 μm. In certain embodiments, the particles have a D50 particle size in the range of from 35 μm to 55 μm. In certain embodiments, the particles have a D50 particle size in the range of from 35 μm to 45 μm. In certain embodiments, the particles have a D50 particle size in the range of from 37 μm to 42 μm. In certain embodiments, the particles have a D50 particle size in the range of from 38 μm to 40 μm. In certain embodiments, the particles have a D50 particle size in the range of from 40 μm to 45 μm. In certain embodiments, the particles have a D50 particle size in the range of from 45 μm to 55 μm. In certain embodiments, the particles have a D50 particle size in the range of from 50 μm to 54 μm. In certain embodiments, the particles have a D50 particle size in the range of from 55 μm to 65 μm. In certain embodiments, the particles have a D50 particle size in the range of from 60 μm to 65 μm. In certain embodiments, the particles have a D50 particle size in the range of from 35 μm to 45 μm. In certain embodiments, the particles have a D50 particle size of about 40 μm. In certain embodiments, the particles have a D50 particle size of about 30 μm, 31 μm, 32 μm, 33 μm, 34 μm, 35 μm, 36 μm, 37 μm, 38 μm, or 39 μm. In certain embodiments, the particles have a D50 particle size of 30 μm 2 μm, 31 m±2 μm, 32 μm 2 μm, 33 m±2 μm, 34 μm±2 m, 35 μm±2 μm, 36 μm±2 m, 37 μm±2 μm, 38 μm±2 μm, or 39 μm±2 μm.

In certain embodiments, the particles have a D90 particle size in the range of from 110 μm to 150 μm. In certain embodiments, the particles have a D90 particle size in the range of from 121 μm to 141 μm. In certain embodiments, the particles have a D90 particle size in the range of from 126 μm to 136 μm. In certain embodiments, the particles have a D90 particle size in the range of from 129 μm to 133 μm. In certain embodiments, the particles have a D90 particle size in the range of from 130 μm to 132 μm. In certain embodiments, the particles have a D90 particle size of about 110 μm, 111 μm, 112 μm, 113 μm, 114 μm, 115 μm, 116 μm, 117 μm, 118 μm, 119 μm, 120 μm, 121 μm, 122 μm, 123 μm, 124 μm, 125 μm, 126 μm, 127 μm, 128 μm, 129 μm, 130 μm, 131 μm, 132 μm, 133 μm, 134 μm, 135 μm, 136 μm, 137 μm, 138 μm, 139 μm, 140 μm, 141 μm, 142 μm, 143 μm, 144 μm, 145 μm, 146 μm, 147 μm, 148 μm, 149 μm, or 150 μm. In certain embodiments, the particles have a D90 particle size of 110 μm±2 μm, 111 μm 2 μm, 112 μm±2 μm, 113 μm 2 μm, 114 μm 2 μm, 115 μm±2 μm, 116 μm 2 μm, 117 μm±2 m, 118 μm 2 μm, 119 μm 2 μm, 120 μm±2 m, 121 μm±2 μm, 122 μm 2 μm, 123 μm 2 μm, 124 μm±2 m, 125 μm±2 μm, 126 μm 2 μm, 127 μm 2 μm, 128 μm±2 m, 129 μm±2 m, 130 μm 2 μm, 131 μm±2 m, 132 μm 2 μm, 133 μm±2 μm, 134 μm 2 μm, 135 μm±2 μm, 136 μm 2 μm, 137 μm 2 μm, 138 μm±2 μm, 139 μm 2 μm, 140 μm±2 m, 141 μm 2 μm, 142 μm 2 μm, 143 μm±2 m, 144 μm 2 μm, 145 μm 2 μm, 146 μm 2 μm, 147 μm±2 m, 148 μm 2 μm, 149 μm±2 m, or 150 m±2 μm. In certain embodiments, the particles have a D90 particle size of 129 μm±2 μm. In certain embodiments, the particles have a D90 particle size of 130 μm±2 μm. In certain embodiments, the particles have a D90 particle size of 131 μm±2 μm. In certain embodiments, the particles have a D90 particle size of 132 μm±2 μm. In certain embodiments, the particles have a D90 particle size of 133 μm±2. In certain embodiments, the particles have a D90 particle size of 129 μm±1 μm. In certain embodiments, the particles have a D90 particle size of 130 μm±1 μm. In certain embodiments, the particles have a D90 particle size of 131 μm±1 μm. In certain embodiments, the particles have a D90 particle size of 132 μm±1 μm. In certain embodiments, the particles have a D90 particle size of 133 μm±1.

In certain embodiments, the particles have a D90 particle size in the range of from 70 μm to 150 μm. In certain embodiments, the particles have a D90 particle size in the range of from 70 μm to 145 μm. In certain embodiments, the particles have a D90 particle size in the range of from 80 μm to 130 μm. In certain embodiments, the particles have a D90 particle size in the range of from 80 μm to 100 μm. In certain embodiments, the particles have a D90 particle size in the range of from 85 μm to 95 μm. In certain embodiments, the particles have a D90 particle size in the range of from 87 μm to 93 μm. In certain embodiments, the particles have a D90 particle size in the range of from 88 μm to 92 μm. In certain embodiments, the particles have a D90 particle size in the range of from 89 μm to 91 μm. In certain embodiments, the particles have a D90 particle size in the range of from 110 μm to 130 μm. In certain embodiments, the particles have a D90 particle size in the range of from 115 μm to 128 μm. In certain embodiments, the particles have a D90 particle size in the range of from 118 μm to 126 μm. In certain embodiments, the particles have a D90 particle size in the range of from 120 μm to 124 μm. In certain embodiments, the particles have a D90 particle size in the range of from 121 μm to 123 μm. In certain embodiments, the particles have a D90 particle size of about 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, or 110 μm. In certain embodiments, the particles have a D90 particle size of 70 μm±2 μm, 71 μm±2 μm, 72 μm±2 μm, 73 μm±2 μm, 74 μm±2 μm, 75 μm±2 μm, 76 μm±2 μm, 77 μm±2 μm, 78 μm±2 μm, 79 μm 2 μm, 80 μm±2 μm, 81 μm±2 μm, 82 μm±2 μm, 83 μm±2 μm, 84 μm±2 μm, 85 μm±2 μm, 86 μm±2 μm, 87 μm±2 μm, 88 μm±2 μm, 89 μm±2 m, 90 μm±2 m, 91 m±2 μm, 92 μm±2 m, 93 μm±2 μm, 94 μm±2 μm, 95 μm±2 μm, 96 μm±2 μm, 97 μm±2 μm, 98 μm±2 μm, 99 m±2 μm, 100 μm±2 μm, 101 μm±2 μm, 102 μm±2 μm, 103 μm±2 μm, 104 μm±2 μm, 105 μm±2 μm, 106 μm±2 m, 107 μm±2 m, 108 μm±2 μm, 109 μm±2 m, or 110 μm±2 m. In certain embodiments, the particles have a D90 particle size of 120 μm±1 μm. In certain embodiments, the particles have a D90 particle size of 122 μm±1 μm. In certain embodiments, the particles have a D90 particle size of 90 μm±2 μm. In certain embodiments, the particles have a D90 particle size of 90 μm±1 μm.

As particle size may be characterized according to the combination of values using D10, D50, and D90, combinations of the D10, D50, and D90 values set forth above are provided. Exemplary such combinations of D10, D50, and D90 values are set forth in the table below.

| Embodiment | | | |
|---|---|---|---|
| No. | D10 Value | D50 Value | D90 Value |
| 1 | 17 μm to 21 μm | 55 μm to 59 μm | 126 μm to 136 μm |
| 2 | 17 μm to 21 μm | 55 μm to 59 μm | 129 μm to 133 μm |
| 3 | 17 μm to 21 μm | 55 μm to 59 μm | 130 μm to 132 μm |
| 4 | 17 μm to 21 μm | 56 μm to 57 μm | 129 μm to 133 μm |
| 5 | 17 μm to 21 μm | 56 μm to 57 μm | 130 μm to 132 μm |
| 6 | 18 μm to 19 μm | 55 μm to 59 μm | 126 μm to 136 μm |
| 7 | 18 μm to 19 μm | 55 μm to 59 μm | 129 μm to 133 μm |
| 8 | 18 μm to 19 μm | 55 μm to 59 μm | 130 μm to 132 μm |
| 9 | 18 μm to 19 μm | 56 μm to 57 μm | 129 μm to 133 μm |
| 10 | 18 μm to 19 μm | 56 μm to 57 μm | 130 μm to 132 μm |
| 11 | 19 μm ± 2 μm | 55 μm to 59 μm | 126 μm to 136 μm |
| 12 | 19 μm ± 2 μm | 56 μm ± 2 μm | 126 μm to 136 μm |
| 13 | 19 μm ± 2 μm | 56 μm ± 2 μm | 131 μm ± 2 μm |
| 14 | about 18 μm | about 56 μm | about 131 μm |
| 15 | about 19 μm | about 56 μm | about 131 μm |
| 16 | about 20 μm | about 56 μm | about 131 μm |
| 17 | about 19 μm | about 55 μm | about 131 μm |
| 18 | about 19 μm | about 57 μm | about 131 μm |
| 19 | about 19 μm | about 56 μm | about 129 μm |
| 20 | about 19 μm | about 56 μm | about 132 μm |

Further Exemplary such combinations of D10, D50, and D90 values are set forth in the table below.

| Embodiment | | | |
|---|---|---|---|
| No. | D10 Value | D50 Value | D90 Value |
| 1 | 20 μm to 30 μm | 55 μm to 65 μm | 115 μm to 128 μm |
| 2 | 20 μm to 30 μm | 60 μm to 65 μm | 115 μm to 128 μm |
| 3 | 24 μm to 28 pm | 55 μm to 65 μm | 115 μm to 128 μm |
| 4 | 24 μm to 28 μm | 60 μm to 65 μm | 115 μm to 128 μm |
| 5 | 24 μm to 28 μm | 55 μm to 65 μm | 121 μm to 123 μm |
| 6 | 24 μm to 28 μm | 60 μm to 65 μm | 121 μm to 123 μm |
| 7 | about 27 μm | about 61 μm | about 120 μm |
| 8 | about 28 μm | about 62 μm | about 120 μm |
| 9 | about 29 μm | about 62 μm | about 120 μm |
| 10 | 5 μm to 10 μm | 35 μm to 45 μm | 85 μm to 95 μm |
| 11 | 6 μm to 9 μm | 40 μm to 45 μm | 85 μm to 95 μm |
| 12 | 5 μm to 10 μm | 35 μm to 45 μm | 89 μm to 91 μm |
| 13 | 6 μm to 9 μm | 40 μm to 45 μm | 89 μm to 91 μm |
| 14 | about 7 μm | about 40 μm | about 90 μm |
| 15 | about 8 μm | about 40 μm | about 90 μm |
| 16 | about 9 μm | about 40 μm | about 90 μm |
| 17 | about 8 μm | about 41 μm | about 90 μm |
| 18 | about 8 μm | about 42 μm | about 90 μm |

In certain embodiments, the particles have a D50 particle size in the range of from 47 μm to 67 μm, and have a D90 particle size in the range of from 110 μm to 150 μm. In certain embodiments, the particles have a D50 particle size in the range of from 52 μm to 62 μm, and have a D90 particle size in the range of from 121 μm to 141 μm. In certain embodiments, the particles have a D50 particle size in the range of from 55 μm to 59 μm, and have a D90 particle size in the range of from 126 μm to 136 μm. In certain embodiments, the particles have a D50 particle size in the range of from 56 μm to 57 μm, and have a D90 particle size in the range of from 129 μm to 133 μm. In certain embodiments, the particles have a D50 particle size in the range of from 56 μm to 57 μm, and have a D90 particle size in the range of from 130 μm to 132 μm.

In certain embodiments, the particles have a D50 particle size in the range of from 47 μm to 67 μm, have a D90 particle size in the range of from 110 μm to 150 μm, and have a D10 particle size in the range of from 12 μm to 26 μm. In certain embodiments, the particles have a D50 particle size in the range of from 52 μm to 62 μm, have a D90 particle size in the range of from 121 μm to 141 μm, and have a D10 particle size in the range of from 14 μm to 24 μm. In certain embodiments, the particles have a D50 particle size in the range of from 55 μm to 59 μm, have a D90 particle size in the range of from 126 μm to 136 μm, and have a D10 particle size in the range of from 16 μm to 22 μm. In certain embodiments, the particles have a D50 particle size in the range of from 56 μm to 57 μm, have a D90 particle size in the range of from 129 μm to 133 μm, and have a D10 particle size in the range of from 17 μm to 21 μm. In certain embodiments, the particles have a D50 particle size in the range of from 56 μm to 57 μm, have a D90 particle size in the range of from 130 μm to 132 μm, and have a D10 particle size in the range of from 18 μm to 19 μm.

### Physical Form of 3-(3-Methoxy-4-((4-methoxybenzyl)oxy)benzyl)-6-(1-methyl-1H-pyrazol-4-yl)-3H-imidazo[4,5-b]pyridin-2-amine

The particles may be further characterized according to physical form of the 3-(3-methoxy-4-((4-methoxybenzyl)oxy)benzyl)-6-(1-methyl-1H-pyrazol-4-yl)-3H-imidazo[4,5-b]pyridin-2-amine. For example, in certain embodiments, the 3-(3-methoxy-4-((4-methoxy-benzyl)oxy)benzyl)-6-(1-methyl-1H-pyrazol-4-yl)-3H-imidazo[4,5-b]pyridin-2-amine is in crystalline form. In certain embodiments, the 3-(3-methoxy-4-((4-methoxybenzyl) oxy)benzyl)-6-(1-methyl-1H-pyrazol-4-yl)-3H-imidazo[4,5-b]pyridin-2-amine is in the form of a hydrate. In certain embodiments, the 3-(3-methoxy-4-((4-methoxy-benzyl)oxy)benzyl)-6-(1-methyl-1H-pyrazol-4-yl)-3H-imidazo[4,5-b]pyridin-2-amine is in the form of a monohydrate (namely, 3-(3-methoxy-4-((4-methoxybenzyl)oxy)benzyl)-6-(1-methyl-1H-pyrazol-4-yl)-3H-imidazo[4,5-b]pyridin-2-amine monohydrate). In certain embodiments, the 3-(3-methoxy-4-((4-methoxy-benzyl)oxy)benzyl)-6-(1-methyl-1H-pyrazol-4-yl)-3H-imidazo[4,5-b]pyridin-2-amine is in the form of a crystalline monohydrate (namely, 3-(3-methoxy-4-((4-methoxybenzyl)oxy)benzyl)-6-(1-methyl-1H-pyrazol-4-yl)-3H-imidazo[4,5-b]pyridin-2-amine monohydrate).

The particles may be further characterized according to the amount of 3-(3-methoxy-4-((4-methoxybenzyl)oxy)benzyl)-6-(1-methyl-1H-pyrazol-4-yl)-3H-imidazo[4,5-b]pyridin-2-amine in the particles that is in the form of plate-like crystals. For example, in certain embodiments, at least 50% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of plate-like crystals. In certain embodiments, at least 75% by weight of the compound 3-(3-methoxy-4-((4-methoxybenzyl)oxy)benzyl)-6-(1-methyl-1H-pyrazol-4-yl)-3H-imidazo[4,5-b]pyridin-2-amine in the particles is in the form of plate-like crystals. In certain embodiments, at least 80% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of plate-like crystals. In certain embodiments, at least 90% by weight of the compound 3-(3-methoxy-4-((4-methoxybenzyl)oxy)benzyl)-6-(1-methyl-1H-pyrazol-4-yl)-3H-imidazo[4,5-b]pyridin-2-amine in the particles is in the form of plate-like crystals. In certain embodiments, at least 95% by weight of the compound 3-(3-methoxy-4-((4-methoxybenzyl)oxy) benzyl)-6-(1-methyl-1H-pyrazol-4-yl)-3H-imidazo[4,5-b]pyridin-2-amine in the particles is in the form of plate-like crystals.

The particles may be further characterized according to the amount of 3-(3-methoxy-4-((4-methoxybenzyl)oxy)benzyl)-6-(1-methyl-1H-pyrazol-4-yl)-3H-imidazo[4,5-b]pyridin-2-amine monohydrate in the particles that is in the form of plate-like crystals. For example, in certain embodiments, at least 50% by weight of the compound 3-(3-methoxy-4-((4-methoxy-benzyl)oxy)benzyl)-6-(1-methyl-1H-pyrazol-4-yl)-3H-imidazo[4,5-b]pyridin-2-amine monohydrate in the particles is in the form of plate-like crystals. In certain embodiments, at least 75% by weight of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate in the particles is in the form of plate-like crystals. In certain embodiments, at least 80% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of plate-like crystals. In certain embodiments, at least 90% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of plate-like crystals. In certain embodiments, at least 95% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of plate-like crystals.

The particles may be further characterized according to the amount of 3-(3-methoxy-4-((4-methoxybenzyl)oxy)benzyl)-6-(1-methyl-1H-pyrazol-4-yl)-3H-imidazo[4,5-b]pyridin-2-amine in the particles that is in the form of needle-like crystals. For example, in certain embodiments, less than 20% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of needle-like crystals. In certain embodiments, less than 10% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of needle-like crystals. In certain embodiments, less than 5% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of needle-like crystals. In certain embodiments, less than 2% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of needle-like crystals. In certain embodiments, less than 1% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine in the particles is in the form of needle-like crystals.

The particles may be further characterized according to the amount of 3-(3-methoxy-4-((4-methoxybenzyl)oxy)benzyl)-6-(1-methyl-1H-pyrazol-4-yl)-3H-imidazo[4,5-b]pyridin-2-amine monohydrate in the particles that is in the form of needle-like crystals. For example, in certain embodiments, less than 20% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of needle-like crystals. In certain embodiments, less than 10% by weight of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate in the particles is in the form of needle-like crystals. In certain embodiments, less than 5% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of needle-like crystals. In certain embodiments, less than 2% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of needle-like crystals. In certain embodiments, less than 1% by weight of the compound 3-(3-methoxy-4-((4-methoxybenzyl) oxy)benzyl)-6-(1-methyl-1H-pyrazol-4-yl)-3H-imidazo[4,5-b]pyridin-2-amine monohydrate in the particles is in the form of needle-like crystals.

### Crystalline Forms of 3-(3-Methoxy-4-((4-methoxybenzyl)oxy)benzyl)-6-(1-methyl-1H-pyrazol-4-yl)-3H-imidazo[4,5-b]pyridin-2-amine Monohydrate

As indicated hereinabove, the 3-(3-methoxy-4-((4-methoxybenzyl)oxy)benzyl)-6-(1-methyl-1H-pyrazol-4-yl)-3H-imidazo[4,5-b]pyridin-2-amine monohydrate may be in crystalline form. The crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate compound may be characterized by X-ray powder diffraction, differential scanning calorimetry, and other spectroscopic techniques. Methods for preparing and using the compound are described herein below.

In certain embodiments, the 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline Form 3. The compound 3-(3-methoxy-4-((4-methoxybenzyl)oxy)benzyl)-6-(1-methyl-1H-pyrazol-4-yl)-3H-imidazo[4,5-b]pyridin-2-amine monohydrate crystalline Form 3 is characterized by exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2.

In certain embodiments, the 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline Form 1. The compound 3-(3-methoxy-4-((4-methoxybenzyl)oxy)benzyl)-6-(1-methyl-1H-pyrazol-4-yl)-3H-imidazo[4,5-b]pyridin-2-amine monohydrate crystalline Form 1 is characterized by exhibiting an X-ray powder diffraction pattern comprising peaks at the following diffraction angles (2θ): 7.1±0.2, 10.4±0.2, 10.7±0.2, 12.4±0.2, 12.7±0.2, and 14.3±0.2.

Each are described in more detail below.

### A. Crystalline Form 3

Accordingly, in certain embodiments, the particles are further characterized by the feature that the compound is 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2. In certain embodiments, the X-ray powder diffraction pattern further comprises a peak at the following diffraction angle (2θ): 16.6±0.2. In certain embodiments, the X-ray powder diffraction pattern further comprises a peak at the following diffraction angle (2θ): 22.6±0.2. In certain embodiments, the X-ray powder diffraction pattern further comprises a peak at the following diffraction angle (2θ): 22.9±0.2. In certain embodiments, the X-ray powder diffraction pattern further comprise a peak at one, two, three, four, or more of the following diffraction angles (2θ): 12.8 0.2, 13.2 0.2, 17.3 0.2, 19.0±0.2, 23.9±0.2, 26.5±0.2, 28.4±0.2, and 28.8±0.2. In certain embodiments, the X-ray powder diffraction pattern further comprises peaks at the following diffraction angles (2θ): 12.8±0.2, 13.2±0.2, 17.3±0.2, 19.0±0.2, 23.9±0.2, 26.5±0.2, 28.4±0.2, and 28.8±0.2.

In certain embodiments, the relative intensity of the peak at said diffraction angles (2θ) is at least 15%. In certain embodiments, the relative intensity of the peak at said diffraction angles (2θ) is at least 20%. In certain embodiments, the relative intensity of the peak at said diffraction angles (2θ) is at least 30%.

In certain embodiments, the compound is characterized by the following X-ray powder diffraction pattern expressed in terms of diffraction angle 20, inter-planar distances d, and relative intensity (expressed as a percentage with respect to the most intense peak):

| Angle [2θ] | d-spacing [Å] | Relative Intensity [%] |
|---|---|---|
| 12.78 | 6.93 | 7.3 |
| 13.19 | 6.71 | 8.1 |
| 14.87 | 5.96 | 100.0 |
| 16.62 | 5.33 | 19.3 |
| 17.34 | 5.12 | 8.9 |
| 19.03 | 4.66 | 11.8 |
| 20.19 | 4.40 | 49.1 |
| 20.67 | 4.30 | 31.0 |
| 21.40 | 4.15 | 40.7 |
| 22.56 | 3.94 | 28.6 |
| 22.90 | 3.88 | 26.1 |
| 23.93 | 3.72 | 14.4 |
| 25.09 | 3.55 | 64.4 |
| 26.53 | 3.36 | 7.4 |
| 28.00 | 3.19 | 41.6 |
| 28.35 | 3.15 | 10.5 |
| 28.83 | 3.10 | 6.2 |
| 29.95 | 2.98 | 42.4 |

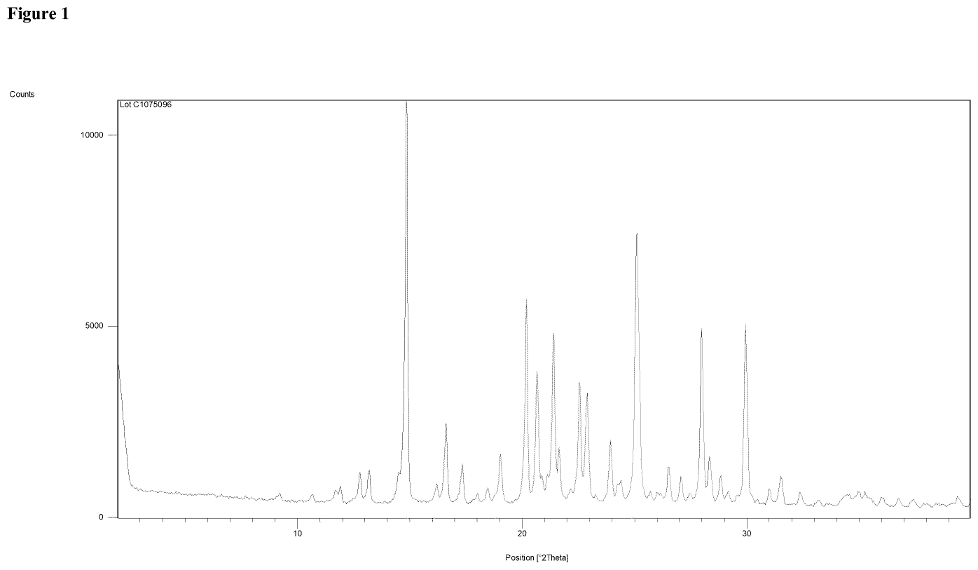

In certain embodiments, the compound has an X-ray powder diffraction pattern that is substantially as shown in FIG. 1.

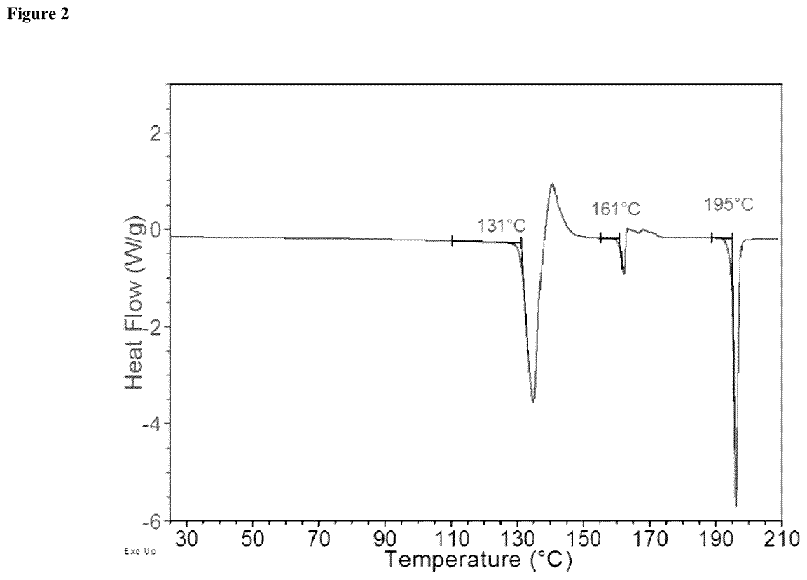

In certain embodiments, the compound has a differential scanning calorimetry curve substantially the same as shown in FIG. 2.

### B. Crystalline Form 1

In certain embodiments, the 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline Form 1. 3-(3-Methoxy-4-((4-methoxybenzyl)oxy)benzyl)-6-(1-methyl-1H-pyrazol-4-yl)-3H-imidazo[4,5-b]pyridin-2-amine monohydrate crystalline Form 1 is characterized by exhibiting an X-ray powder diffraction pattern comprising peaks at the following diffraction angles (2θ): 7.1 ±0.2, 10.4±0.2, 10.7±0.2, 12.4±0.2, 12.7±0.2, and 14.3±0.2. The crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate compound may be characterized by X-ray powder diffraction, differential scanning calorimetry, and other spectroscopic techniques. Crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 is described in international patent application publication WO 2016/100677, and methods for using the compound are described herein below.

Accordingly, in certain embodiments, the particles are further characterized by the feature that the compound is 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 7.1±0.2, 10.4±0.2, 10.7±0.2, 12.4±0.2, 12.7±0.2, and 14.3±0.2. In certain embodiments, the X-ray powder diffraction pattern further comprises a peak at the following diffraction angle (2θ): 8.9±0.2. In certain embodiments, the X-ray powder diffraction pattern further comprises a peak at the following diffraction angle (2θ): 3.6±0.2.

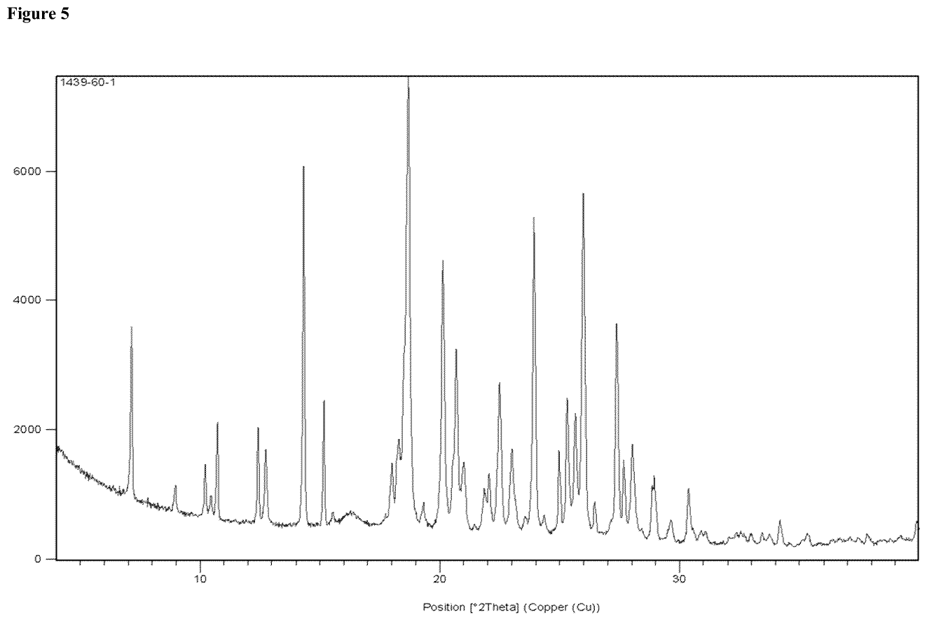

In certain embodiments, the compound has an X-ray powder diffraction pattern that is substantially as shown in FIG. 5.

### II. Therapeutic Applications of the Particles and Pharmaceutical Compositions

The particles described herein may be used to treat inflammatory disease, autoimmune disease, pain, osteoarthritis, defect of bone metabolism, and cancer. Accordingly, one aspect of the invention provides a method of treating a disease or condition selected from the group consisting of an inflammatory disease, autoimmune disease, pain, osteoarthritis, defect of bone metabolism, and cancer. The method comprises administering a therapeutically effective amount of particles described herein to a subject in need thereof to treat the disease or condition. The compound and/or particles may be formulated as a pharmaceutical composition. Preferably, the particles contain the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2. In certain embodiments, the X-ray powder diffraction pattern further comprises a peak at the following diffraction angle (2θ): 16.6±0.2. In certain embodiments, the X-ray powder diffraction pattern further comprises a peak at the following diffraction angle (2θ): 22.6±0.2. In certain embodiments, the X-ray powder diffraction pattern further comprises a peak at the following diffraction angle (2θ): 22.9±0.2. In certain embodiments, the X-ray powder diffraction pattern further comprises peaks at the following diffraction angles (2θ): 12.8±0.2, 13.2 0.2, 17.3 0.2, 19.0±0.2, 23.9±0.2, 26.5±0.2, 28.4±0.2, and 28.8±0.2.

In certain embodiments, the disease or condition is an inflammatory disease. In certain embodiments, the disease or condition is autoimmune disease. In certain embodiments, the disease or condition is pain. In certain embodiments, the disease or condition is pain due to osteoarthritis. In certain embodiments, the disease or condition is joint pain due to osteoarthritis, such as knee joint pain due to osteoarthritis. In certain embodiments, the disease or condition is post-operative pain. In certain embodiments, the disease or condition is osteoarthritis. In certain embodiments, the disease or condition is cancer.

In certain embodiments, the subject is a human.

Another aspect of the invention provides a method of inhibiting the activity of a tropomyosin-related kinase. The method comprises contacting a tropomyosin-related kinase with an effective amount of particles described herein to inhibit the activity of said tropomyosin-related kinase. In certain embodiments, the tropomyosin-related kinase is tropomyosin-related kinase A. In certain embodiments, the tropomyosin-related kinase is tropomyosin-related kinase B. In certain embodiments, the tropomyosin-related kinase is tropomyosin-related kinase C. Preferably, the particles contain the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2. In certain embodiments, the X-ray powder diffraction pattern further comprises a peak at the following diffraction angle (2θ): 16.6±0.2. In certain embodiments, the X-ray powder diffraction pattern further comprises a peak at the following diffraction angle (2θ): 22.6±0.2. In certain embodiments, the X-ray powder diffraction pattern further comprises a peak at the following diffraction angle (2θ): 22.9±0.2. In certain embodiments, the X-ray powder diffraction pattern further comprises peaks at the following diffraction angles (2θ): 12.8±0.2, 13.2 0.2, 17.3 0.2, 19.0±0.2, 23.9±0.2, 26.5±0.2, 28.4±0.2, and 28.8±0.2.

Another aspect of the invention provides a method of inhibiting the activity of a cellular receptor for colony stimulating factor-1. The method comprises contacting said cellular receptor for colony stimulating factor-1 with an effective amount of particles described herein to inhibit the activity of said cellular receptor for colony stimulating factor-1. Preferably, the particles contain the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2. In certain embodiments, the X-ray powder diffraction pattern further comprises a peak at the following diffraction angle (2θ): 22.6±0.2. In certain embodiments, the X-ray powder diffraction pattern further comprises a peak at the following diffraction angle (2θ): 22.9±0.2. In certain embodiments, the X-ray powder diffraction pattern further comprises peaks at the following diffraction angles (2θ): 12.8±0.2, 13.2±0.2, 17.3±0.2, 19.0±0.2, 23.9±0.2, 26.5±0.2, 28.4±0.2, and 28.8±0.2.

Another aspect of the invention provides for the use of particles described herein in the manufacture of a medicament. In certain embodiments, the medicament is for treating a disorder described herein, such as pain.

Another aspect of the invention provides for the use of particles described herein for treating a medical disorder, such a medical disorder described herein (e.g., pain).

### III. Methods of Preparing Particles

Another aspect of the invention provides methods for preparing particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate, such as particles having a D50 particle size in the range of from 40 μm to 75 μm. The method generally entails crystallizing 3-(3-methoxy-4-((4-methoxybenzyl)oxy)benzyl)-6-(1-methyl-1H-pyrazol-4-yl)-3H-imidazo[4,5-b]pyridin-2-amine monohydrate from a solution containing acetone, water, and 3-(3-methoxy-4-((4-methoxybenzyl) oxy)benzyl)-6-(1-methyl-1H-pyrazol-4-yl)-3H-imidazo[4,5-b]pyridin-2-amine according to a particular temperature gradient, then heating the resulting mixture to a temperature of about 50° C., and then cooling the mixture across multiple temperatures while holding for certain durations of time at certain temperature plateaus, then heating the resulting mixture to a temperature of about 50° C., and then cooling the mixture across multiple temperatures while holding for certain durations of time at certain temperature plateaus, and then heating the resulting mixture to a temperature of about 50° C., then cooling the mixture across multiple temperatures while holding for certain durations of time at certain temperature plateaus, and then optionally heating the resulting mixture to a temperature of about 50° C., then cooling the mixture across multiple temperatures while holding for certain durations of time at certain temperature plateaus.

Accordingly, one aspect of the invention provides a method of preparing particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm, the method comprising the steps of.
a. admixing (i) a first solution having a temperature of about 50° C. and containing acetone, water, and 3-(3-methoxy-4-((4-methoxybenzyl)oxy)benzyl)-6-(1-methyl-1H-pyrazol-4-yl)-3H-imidazo[4,5-b]pyridin-2-amine and (ii) an aliquot of 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form to provide a first mixture;
b. maintaining the first mixture at a temperature in the range of from 42° C. to 48° C. for a duration of about 1 hour, then cooling the first mixture to a temperature in the range of from 37° C. to 42° C. and maintaining the first mixture at a temperature in the range of from 37° C. to 42° C. for about 1 hour, then cooling the first mixture to a temperature in the range of from 2° C. to 8° C. and maintaining the first mixture at a temperature in the range of from 2° C. to 8° C. for about 1 hour, to produce a second mixture;
c. heating the second mixture to a temperature in the range of from 48° C. to 53° C., maintaining the second mixture at a temperature in the range of from 48° C. to 53° C. for about 3 hours, then cooling the second mixture to a temperature in the range of from 43° C. to 47° C. and maintaining the second mixture at a temperature in the range of from 43° C. to 47° C. for about 1 hour, then cooling the second mixture to a temperature in the range of from 37° C. to 42° C. and maintaining the second mixture at a temperature in the range of from 37° C. to 42° C. for about 1 hour, then cooling the second mixture to a temperature in the range of from 2° C. to 8° C. and maintaining the second mixture at a temperature in the range of from 2° C. to 8° C. for about 1 hour, to produce a third mixture;
d. heating the third mixture to a temperature in the range of from 48° C. to 53° C., maintaining the third mixture at a temperature in the range of from 48° C. to 53° C. for about 3 hours, then cooling the third mixture to a temperature in the range of from 43° C. to 47° C. and maintaining the third mixture at a temperature in the range of from 43° C. to 47° C. for about 1 hour, then cooling the third mixture to a temperature in the range of from 37° C. to 42° C. and maintaining the third mixture at a temperature in the range of from 37° C. to 42° C. for about 1 hour, then cooling the third mixture to a temperature in the range of from 2° C. to 8° C. and maintaining the third mixture at a temperature in the range of from 2° C. to 8° C. for about 1 hour, to produce a crystallization mixture; and
e. isolating said particles from the crystallization mixture.

In certain embodiment, the method further comprises between step (d) and (e), heating the crystallization mixture to a temperature in the range of from 48° C. to 53° C., maintaining the crystallization mixture at a temperature in the range of from 48° C. to 53° C. for about 3 hours, then cooling the crystallization mixture to a temperature in the range of from 43° C. to 47° C. and maintaining the crystallization mixture at a temperature in the range of from 43° C. to 47° C. for about 1 hour, then cooling the crystallization mixture to a temperature in the range of from 37° C. to 42° C. and maintaining the crystallization mixture at a temperature in the range of from 37° C. to 42° C. for about 1 hour, then cooling the crystallization mixture to a temperature in the range of from 2° C. to 8° C. and maintaining the crystallization mixture at a temperature in the range of from 2° C. to 8° C. for about 1 hour.

In certain embodiments, the method further comprises the step of: between step (d) and (e), heating the crystallization mixture to a temperature of 50° C., maintaining the crystallization mixture at a temperature of 50° C. for about 3 hours, then cooling the crystallization mixture to a temperature of 45° C. and maintaining the crystallization mixture at a temperature of 45° C. for about 1 hour, then cooling the crystallization mixture to a temperature of 40° C. and maintaining the crystallization mixture at a temperature of 40° C. for about 1 hour, then cooling the crystallization mixture to a temperature of 5° C. and maintaining the crystallization mixture at a temperature of 5° C. for about 1 hour.

In certain embodiments, the method further comprises the step of: between step (d) and (e), heating the crystallization mixture to a temperature in the range of from 48° C. to 53° C., maintaining the crystallization mixture at a temperature in the range of from 48° C. to 53° C. for about 3 hours, then cooling the crystallization mixture to a temperature in the range of from 43° C. to 47° C. and maintaining the crystallization mixture at a temperature in the range of from 43° C. to 47° C. for about 1 hour, then cooling the crystallization mixture to a temperature in the range of from 37° C. to 42° C. and maintaining the crystallization mixture at a temperature in the range of from 37° C. to 42° C. for about 1 hour, then cooling the crystallization mixture to a temperature in the range of from 2° C. to 8° C. and maintaining the crystallization mixture at a temperature in the range of from 2° C. to 8° C. for about 1 hour, then heating the crystallization mixture to a temperature in the range of from 48° C. to 53° C., maintaining the crystallization mixture at a temperature in the range of from 48° C. to 53° C. for about 3 hours, then cooling the crystallization mixture to a temperature in the range of from 43° C. to 47° C. and maintaining the crystallization mixture at a temperature in the range of from 43° C. to 47° C. for about 1 hour, then cooling the crystallization mixture to a temperature in the range of from 37° C. to 42° C. and maintaining the crystallization mixture at a temperature in the range of from 37° C. to 42° C. for about 1 hour, then cooling the crystallization mixture to a temperature in the range of from 2° C. to 8° C. and maintaining the crystallization mixture at a temperature in the range of from 2° C. to 8° C. for about 1 hour.

In certain embodiments, the method further comprises the step of: between step (d) and (e), heating the crystallization mixture to a temperature of 50° C., maintaining the crystallization mixture at a temperature of 50° C. for about 3 hours, then cooling the crystallization mixture to a temperature of 45° C. and maintaining the crystallization mixture at a temperature of 45° C. for about 1 hour, then cooling the crystallization mixture to a temperature of 40° C. and maintaining the crystallization mixture at a temperature of 40° C. for about 1 hour, then cooling the crystallization mixture to a temperature of 5° C. and maintaining the crystallization mixture at a temperature of 5° C. for about 1 hour, then heating the crystallization mixture to a temperature of 50° C., maintaining the crystallization mixture at a temperature of 50° C. for about 3 hours, then cooling the crystallization mixture to a temperature of 45° C. and maintaining the crystallization mixture at a temperature of 45° C. for about 1 hour, then cooling the crystallization mixture to a temperature of 40° C. and maintaining the crystallization mixture at a temperature of 40° C. for about 1 hour, then cooling the crystallization mixture to a temperature of 5° C. and maintaining the crystallization mixture at a temperature of 5° C. for about 1 hour.

In certain embodiments, the ratio of acetone to water in the first solution is about 5:1 by volume.

In certain embodiments, step (a) comprises admixing (i) a first solution having a temperature of about 50° C. and containing acetone, water, and 3-(3-methoxy-4-((4-methoxybenzyl)oxy)benzyl)-6-(1-methyl-1H-pyrazol-4-yl)-3H-imidazo[4,5-b]pyridin-2-amine and (ii) an aliquot of 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2 to provide a first mixture.

In certain embodiments, step (b) comprises maintaining the first mixture at a temperature of 45° C. for a duration of about 1 hour, then cooling the first mixture to a temperature of 40° C. and maintaining the first mixture at a temperature of 40° C. for about 1 hour, then cooling the first mixture to a temperature of 5° C. and maintaining the first mixture at a temperature of 5° C. for about 1 hour, to produce a second mixture.

In certain embodiments, step (c) comprises heating the second mixture to a temperature of 50° C., maintaining the second mixture at a temperature of 50° C. for about 3 hours, then cooling the second mixture to a temperature of 45° C. and maintaining the second mixture at a temperature of 45° C. for about 1 hour, then cooling the second mixture to a temperature of 40° C. and maintaining the second mixture at a temperature of 40° C. for about 1 hour, then cooling the second mixture to a temperature of 5° C. and maintaining the second mixture at a temperature of 5° C. for about 1 hour, to produce a third mixture.

In certain embodiments, step (d) comprises heating the third mixture to a temperature of 50° C., maintaining the third mixture at a temperature of 50° C. for about 3 hours, then cooling the third mixture to a temperature of 45° C. and maintaining the third mixture at a temperature of 45° C. for about 1 hour, then cooling the third mixture to a temperature of 40° C. and maintaining the third mixture at a temperature of 40° C. for about 1 hour, then cooling the third mixture to a temperature of 5° C. and maintaining the third mixture at a temperature of 5° C. for about 1 hour, to produce a crystallization mixture.

Another aspect of the invention provides a method of preparing particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm, the method comprising the steps of:
a. admixing (i) a first solution having a temperature of about 50° C. and containing acetone, water, and 3-(3-methoxy-4-((4-methoxybenzyl)oxy)benzyl)-6-(1-methyl-1H-pyrazol-4-yl)-3H-imidazo[4,5-b]pyridin-2-amine and (ii) an aliquot of 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form to provide a first mixture;
b. maintaining the first mixture at a temperature in the range of from 42° C. to 48° C. for a duration of about 1 hour, then cooling the first mixture to a temperature in the range of from 37° C. to 42° C. and maintaining the first mixture at a temperature in the range of from 37° C. to 42° C. for about 1 hour, then cooling the first mixture to a temperature in the range of from 2° C. to 8° C. and maintaining the first mixture at a temperature in the range of from 2° C. to 8° C. for about 1 hour, to produce a second mixture;
c. heating the second mixture to a temperature in the range of from 48° C. to 53° C., maintaining the third mixture at a temperature in the range of from 48° C. to 53° C. for about 3 hours, then cooling the third mixture to a temperature in the range of from 43° C. to 47° C. and maintaining the third mixture at a temperature in the range of from 43° C. to 47° C. for about 1 hour, then cooling the third mixture to a temperature in the range of from 37° C. to 42° C. and maintaining the third mixture at a temperature in the range of from 37° C. to 42° C. for about 1 hour, then cooling the third mixture to a temperature in the range of from 2° C. to 8° C. and maintaining the third mixture at a temperature in the range of from 2° C. to 8° C. for about 1 hour, to produce a crystallization mixture; and
d. isolating said particles from the crystallization mixture.

In certain embodiments, the ratio of acetone to water in the first solution is about 5:1 by volume.

In certain embodiments, step (a) comprises admixing (i) a first solution having a temperature of about 50° C. and containing acetone, water, and 3-(3-methoxy-4-((4-methoxybenzyl)oxy)benzyl)-6-(1-methyl-1H-pyrazol-4-yl)-3H-imidazo[4,5-b]pyridin-2-amine and (ii) an aliquot of 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2±0.2, 20.7±0.2, 21.4±0.2, 25.1±0.2, 28.0±0.2, and 30.0±0.2 to provide a first mixture.

In certain embodiments, step (b) comprises maintaining the first mixture at a temperature of 45° C. for a duration of about 1 hour, then cooling the first mixture to a temperature of 40° C. and maintaining the first mixture at a temperature of 40° C. for about 1 hour, then cooling the first mixture to a temperature of 5° C. and maintaining the first mixture at a temperature of 5° C. for about 1 hour, to produce a second mixture.

### IV. Pharmaceutical Compositions and Dosing Considerations

As indicated above, the invention provides pharmaceutical compositions, which comprise a therapeutically-effective amount of particles described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising particles described herein and a pharmaceutically acceptable carrier.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising particles described herein in a therapeutically effective amount for the treatment of a medical disease or condition described herein.

In a more specific embodiment, the invention provides a pharmaceutical composition, comprising (i) a particle described herein and (ii) a pharmaceutically acceptable excipient.

In certain embodiments, the pharmaceutically acceptable excipient comprises a diluent. In certain embodiments, the diluent is selected from: malitol, sunflower oil, ammonium alginate, calcium carbonate, calcium lactate, calcium phosphate dibasic anhydrous, dibasic calcium phosphate dihydrate, calcium phosphate tribasic, calcium silicate, calcium sulfate, cellulose (powdered, silicified microcrystalline), cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, lactose (anhydrous, monohydrate and corn starch, monohydrate and microcrystalline cellulose, spray dried), magnesium carbonate, magnesium oxide, maltodextrin, maltose, mannitol, medium-chain triglycerides, microcrystalline cellulose, polydextrose, polymethacrylates, simethicone, sodium alginate, sodium chloride, sorbitol, starch (pregelatinized, sterilizable maize), sucrose, sugar spheres, sulfobutylether b-cyclodextrin, talc, tragacanth, trehalose, and xylitol.

In certain embodiments, the pharmaceutically acceptable excipient comprises a suspending agent. In certain embodiments, the suspending agent is selected from: acacia, agar, alginic acid, bentonite, calcium stearate, carbomer, carboxymethylcellulose (calcium and sodium), carrageenan, cellulose (microcrystalline, microcyrstalline and carboxymethylcellulose sodium, powdered), colloidal silicone dioxide, destrin, gelatin, guar gum, hectorite, hydrophobic colloidal silica, hydroxyethyl cellulose, hydroxymethyl cellulose, hydroxypropyl cellulose, hypromellose, kaolin, magnesium aluminum silicate, maltitol solutions, medium-chain triglycerides, methylcellulose, phenylmercuric borate, phospholipids, poycarbophil, polyethylene glycol, polyoxyethylene sorbitan fatty acid esters, povidone (polyvinylpyrrrolidone), propylene glycol alginate, saponite, sesame oil, sodium alginate, sorbitan esters, sucrose, tragacanth, vitamin E polyethylene glycol succinate, and xanthan gum.

In certain embodiments, the pharmaceutically acceptable excipient comprises a buffering agent. In certain embodiments, the buffering agent is selected from: adipic acid, ammonia solution, boric acid, calcium carbonate, calcium hydroxide, calcium lactate, calcium phosphate, calcium phosphate tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine

In certain embodiments, the pharmaceutically acceptable excipient comprises a diluent, suspending agent, and buffering agent, wherein the diluent is sorbitol, the suspending agent is povidone, and the buffering agent is phosphoric acid.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Preparation of Crystalline 3-(3-methoxy-4-((4-methoxybenzyl)oxy) benzyl)-6-(1-methyl-1H-pyrazol-4-yl)-3H-imidazo[4,5-b]pyridin-2-amine Monohydrate Form 3

The title compound was prepared according to the following procedure. A suspension of sesquihydrate 3-(3-methoxy-4-((4-methoxybenzyl)oxy)benzyl)-6-(1-methyl-1H-pyrazol-4-yl)-3H-imidazo[4,5-b]pyridin-2-amine in a mixture of acetone and water (where the mixture was 80/20 w/w acetone to water) was heated at a temperature of 50° C., without stirring. Thereafter, from the resulting mixture, crystalline 3-(3-methoxy-4-((4-methoxybenzyl)oxy) benzyl)-6-(1-methyl-1H-pyrazol-4-yl)-3H-imidazo[4,5-b]pyridin-2-amine monohydrate Form 3 was isolated.

The sesquihydrate 3-(3-methoxy-4-((4-methoxybenzyl)oxy)benzyl)-6-(1-methyl-1H-pyrazol-4-yl)-3H-imidazo[4,5-b]pyridin-2-amine starting material used in this example is described in international patent application publication WO 2016/100677.

### Example 2—Preparation of Crystalline 3-(3-methoxy-4-((4-methoxybenzyl)oxy) benzyl)-6-(1-methyl-1H-pyrazol-4-yl)-3H-imidazo[4,5-b]pyridin-2-amine Monohydrate Form 3

The title compound was prepared according to the following procedure. 3-(3-Methoxy-4-((4-methoxybenzyl)oxy)benzyl)-6-(1-methyl-1H-pyrazol-4-yl)-3H-imidazo[4,5-b]pyridin-2-amine was dissolved in 80/20 w/w mixture of acetone and water heated to a temperature of 70° C. The resulting solution was cooled to a temperature of 55° C. and then filtered through a 0.2 μm cartridge. The filtered solution was cooled to a temperature of 50° C. (where cooling was performed at a rate of −10° C. per hour), and then solution was seeded with 2% by weight crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3. The resulting mixture was maintained at 50° C. for at least 3 hours, and then two cooling/heating cycles were performed to increase the size of the crystals. Each heating/cooling cycle involved first cooling the mixture to 0° C. (where cooling was performed at a rate of −5° C. per hour), holding the temperature of the mixture at 0° C. for 1 hour, then heating the mixture to a temperature of 50° C. After completing the two cooling/heating cycles, crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 was isolated from the mixture using a filter dryer, washed twice with an 80/20 w/w mixture of acetone/water, and then dried under vacuum for at least 4 hours while maintaining the temperature below 35° C. to provide the final material that is crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3.

### Example 3—Physical Characterization of Crystalline 3-(3-methoxy-4-((4-methoxybenzyl)oxy)benzyl)-6-(1-methyl-1H-pyrazol-4-yl)-3H-imidazo[4,5-b]pyridin-2-amine Monohydrate Form 3

Crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 was characterized by X-ray powder diffraction, single crystal X-ray diffraction, differential scanning calorimetry, dynamic vapor sorption, thermogravimetric analysis/mass spectrometry (TGA-MS), and particle morphology. Results are provided below.

### X-ray Powder Diffraction

An X-ray powder diffractogram taken on crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 is provided in FIG. 1. Tabulated characteristics of the X-ray powder diffractogram in FIG. 1 are provided in the following table, which lists diffraction angle 20, inter-planar distances d, and relative intensity (expressed as a percentage with respect to the most intense peak):

| Angle [2θ] | d-spacing [Å] | Relative Intensity [%] |
|---|---|---|
| 12.78 | 6.93 | 7.3 |
| 13.19 | 6.71 | 8.1 |
| 14.87 | 5.96 | 100.0 |
| 16.62 | 5.33 | 19.3 |
| 17.34 | 5.12 | 8.9 |
| 19.03 | 4.66 | 11.8 |
| 20.19 | 4.40 | 49.1 |
| 20.67 | 4.30 | 31.0 |
| 21.40 | 4.15 | 40.7 |
| 22.56 | 3.94 | 28.6 |
| 22.90 | 3.88 | 26.1 |
| 23.93 | 3.72 | 14.4 |
| 25.09 | 3.55 | 64.4 |
| 26.53 | 3.36 | 7.4 |
| 28.00 | 3.19 | 41.6 |
| 28.35 | 3.15 | 10.5 |
| 28.83 | 3.10 | 6.2 |
| 29.95 | 2.98 | 42.4 |

### Single Crystal X-ray Diffraction

A crystal of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 was characterized by single crystal X-ray diffraction. The crystal structure was determined by XRSCD at 27° C. A monoclinic lattice was determined, with space group C2/c. Lattice parameters of the crystal are the following:

Monoclinic Pn

| | | | | |
|---|---|---|---|---|
| a= | 19.35 | Å | α = γ = | 90° |
| b= | 10.76 | Å | β= | 102.40° |
| c= | 31.92 | Å | | |
| V= | 6485 | Å³ | d= | 1.12 |

### Differential Scanning Calorimetry

A differential scanning calorimetry curve of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 is provided in FIG. 2. The mass loss at temperature 130° C. corresponds to departure of 1 molecule of water.

### Dynamic Vapor Sorption

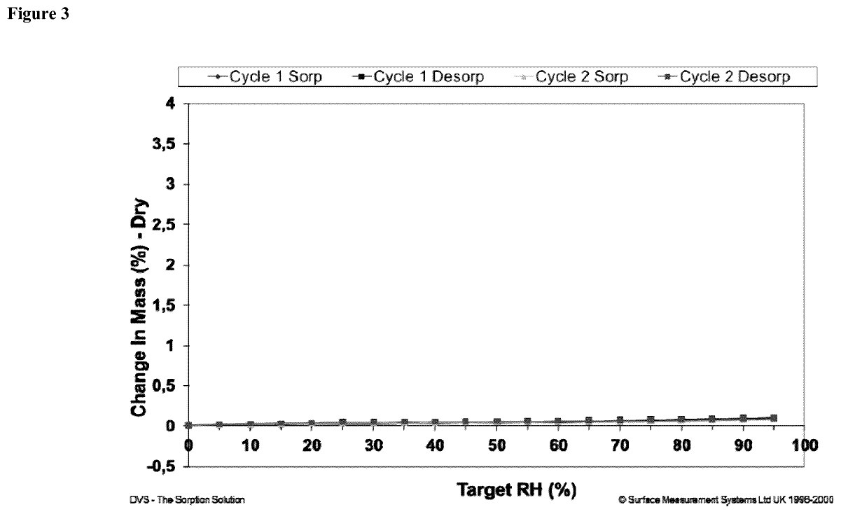

A dynamic vapor sorption isotherm plot of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 is provided in FIG. 3. The dynamic vapor sorption isotherm plot results are from the procedure where after a drying stage at 0% relative humidity, compound sample was exposed to 2 cycles of increasing and decreasing relative humidity at 25° C., with steps of 5% relative humidity, where mass of the sample was recorded throughout the experiment.

The results demonstrate that crystalline 3-(3-methoxy-4-((4-methoxybenzyl)oxy) benzyl)-6-(1-methyl-1H-pyrazol-4-yl)-3H-imidazo[4,5-b]pyridin-2-amine monohydrate Form 3 is not hygroscopic.

### Thermogravimetric Analysis/Mass Spectrometry (TGA-MS)

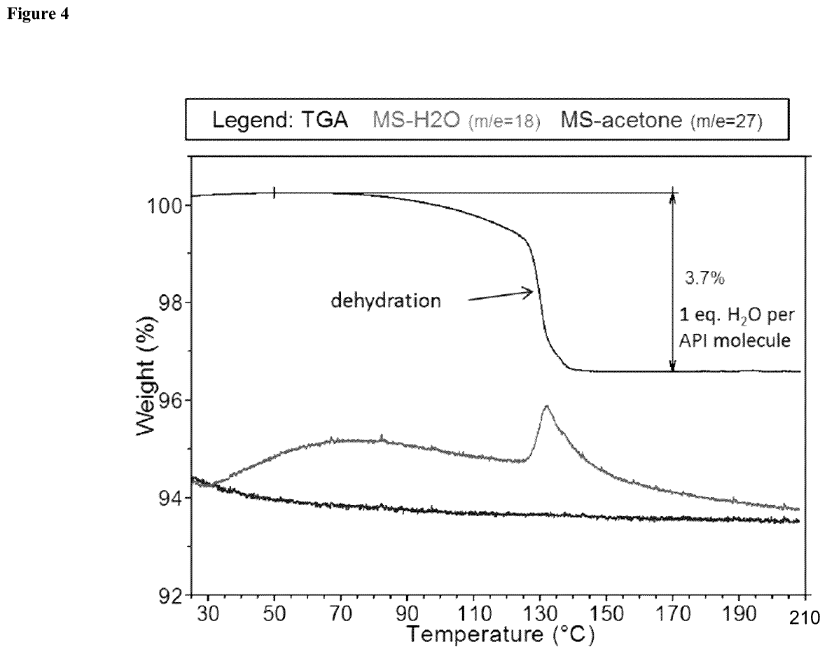

The TGA-MS profile of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 is provided in FIG. 4. The mass profile exhibited a step at 130° C. The mass loss of 3.7% corresponds to departure of one molecule of water per 3-(3-methoxy-4-((4-methoxybenzyl)oxy) benzyl)-6-(1-methyl-1H-pyrazol-4-yl)-3H-imidazo[4,5-b]pyridin-2-amine, and reflects dehydration of the 3-(3-methoxy-4-((4-methoxybenzyl)oxy)benzyl)-6-(1-methyl-1H-pyrazol-4-yl)-3H-imidazo[4,5-b]pyridin-2-amine monohydrate.

### Particle Morphology

Particles of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 were determined to include particles having a rod shape. Rod-shaped particles provide the advantage of better flowability in dry particulate form. The rod-shaped particles also provide the advantage of better filtration when filtering a liquid suspension containing said particles.

### Example 4—Comparison of Stability of Crystalline 3-(3-methoxy-4-((4-methoxybenzyl)oxy)benzyl)-6-(1-methyl-1H-pyrazol-4-yl)-3H-imidazo[4,5-b]pyridin-2-amine Monohydrate Form 3 to Crystalline 3-(3-methoxy-4-((4-methoxybenzyl)oxy)benzyl)-6-(1-methyl-1H-pyrazol-4-yl)-3H-imidazo[4,5-b]pyridin-2-amine Monohydrate Form 1

Stability of crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 was compared to crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1. The physical property analyzed was temperature of dehydration. Crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 is that described in international patent application publication WO 2016/100677.

Crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 was observed to undergo dehydration at room temperature under a stream of nitrogen gas.

By contrast, crystalline 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 did not undergo dehydration until heated to a temperature of 130° C.

### Example 5—Preparation of and Analysis of Particles of 3-(3-Methoxy-4-((4-methoxybenzyl)oxy)benzyl)-6-(1-methyl-1H-pyrazol-4-yl)-3H-imidazo[4,5-b]pyridin-2-amine Monohydrate Form 3

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 were prepared according to the procedure set forth below. The particles were characterized by XRPD, polarized light microscopy, differential scanning calorimetry, thermogravimetric analysis, and particle size analysis.

### Part I—Preparation of Particles of 3-(3-Methoxy-4-((4-methoxybenzyl)oxy)benzyl)-6-(1-methyl-1H-pyrazol-4-yl)-3H-imidazo[4,5-b]pyridin-2-amine Monohydrate Form 3

To an aliquot (9.04 g) of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 that had been micronized was added acetone/water (5:1, v/v, 565 mL) at room temperature. The resulting slurry was heated and stirred with overhead stirring at 55° C. for 1 hour. The resulting clear solution was cooled to 50° C., seeded with crystals of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 (2.8% wt, 250 mg) at 50° C. and stirred for 3 hours, then cooled to 45° C. and stirred for 1 hour, then cooled to 40° C. and stirred for 1 hour, and then cooled to 5° C. and stirred for 1 hour. The slurry was stirred with temperature control (initially heating the slurry to 50° C. and hold for 3 hours, then cooled to 45° C. and hold for 1 hour, then cooled to 40° C. and hold for 1 hour, then cooled 5° C. and hold for 1 hour), whereby this temperature control procedure was performed four times. Then, the slurry was isolated and air-dried for 3 days to provide particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3.

### Part II—Physical Characterization of Particles of 3-(3-Methoxy-4-((4-methoxybenzyl) oxy)benzyl)-6-(1-methyl-1H-pyrazol-4-yl)-3H-imidazo[4,5-b]pyridin-2-amine Monohydrate Form 3

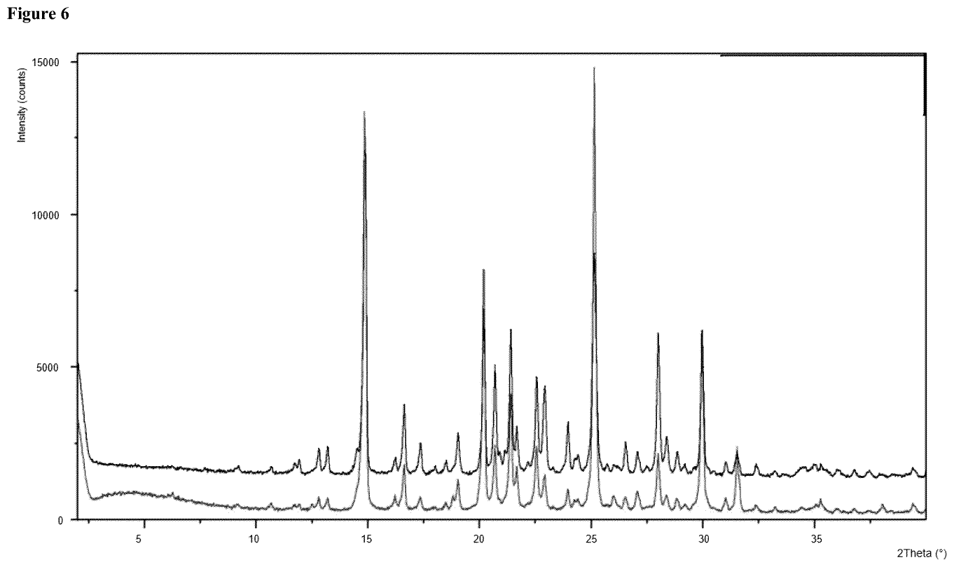

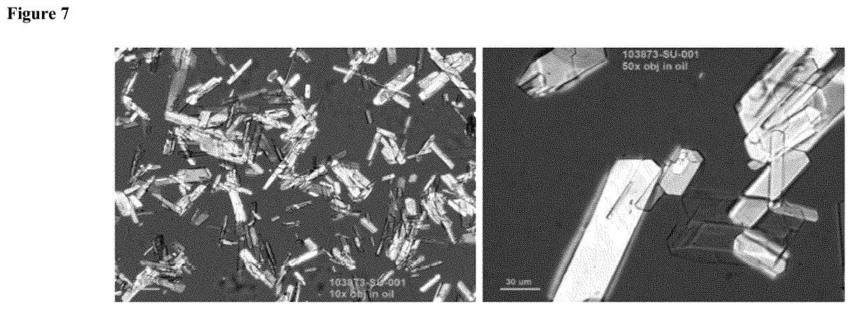

The particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 prepared in Part I above were characterized by XRPD, polarized light microscopy, differential scanning calorimetry, thermogravimetric analysis, and particle size analysis. An X-ray powder diffractogram taken on the particles is provided in FIG. 6. A polarized light microscopy image was obtained on the particles and is provided in FIG. 7. The polarized light microscopy image shows that the particles were quite homogenous.

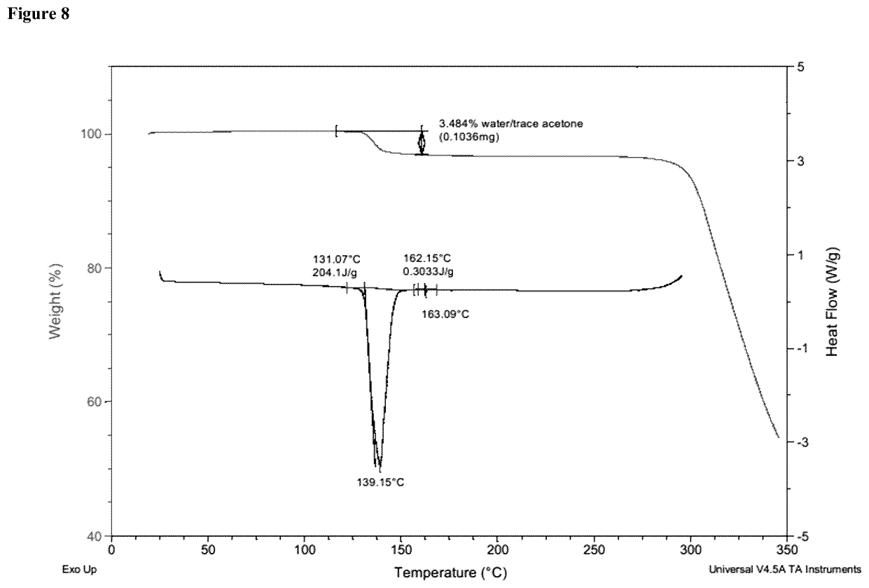

A differential scanning calorimetry curve of the particles is provided in FIG. 8. The mass loss at temperature 131° C. corresponds to departure of 1 molecule of water. Also provided in FIG. 8 is a thermogravimetric analysis profile of the particles. The mass profile exhibited a step at 130° C. The mass loss of 3.5% corresponds to departure of one molecule of water per 3-(3-methoxy-4-((4-methoxybenzyl)oxy)benzyl)-6-(1-methyl-1H-pyrazol-4-yl)-3H-imidazo[4,5-b]pyridin-2-amine, and reflects dehydration of the 3-(3-methoxy-4-((4-methoxybenzyl)oxy) benzyl)-6-(1-methyl-1H-pyrazol-4-yl)-3H-imidazo[4,5-b]pyridin-2-amine monohydrate.

Particle size analysis was performed on the particles using a Malvern Mastersizer 2000 particle size analyzer equipped with a Hydro2000S sample dispersion unit. Instrument parameters and experimental conditions are set forth below:

| | |
|---|---|
| Analysis Model: General Purpose | Particle shape: Irregular |
| Sensitivity: Normal | Absorption: 0.100 |
| Particle RI: 1.520 | Ultrasonics: None |
| Dispersant RI: 1.330 | Stir Speed: 2500 RPM |
| Accessory: Hydro2000S | Obscuration: 10-30% |
| Measurement Time: 10 seconds | Background Time: 10 seconds |
| Measurement Snaps: 10,000 snaps | Background Snaps: 10,000 snaps |

A 50 mg±5 mg of sample of particles was weighed and placed in a 20 mL plastic blood dilution vial. Five drops of dispersant (0.2% (w/v) Igepal in RO water) was added to the vial and mixed to a slurry using the flat end of a spatula. Dispersant (20 mL) was then added and stirred with a spatula to begin dispersion. The sample vial was placed onto a magnetic stir plate and stirred for 5 minutes. While stirring, we withdrew an aliquot of the dispersed sample using a transfer pipet and transferred it to the Hydro2000S until an obscuration of 10-30% was obtained. The sample was allowed to recirculate for 5 minutes at 2500 RPM prior to initiating the sample measurement.

Results of the particle size analysis for particles of 3-(3-methoxy-4-((4-methoxy-benzyl)oxy)benzyl)-6-(1-methyl-1H-pyrazol-4-yl)-3H-imidazo[4,5-b]pyridin-2-amine monohydrate Form 3 prepared according to the procedure in Part I above are set forth in the following table.

| Particle Size | | | |
|---|---|---|---|
| Parameter | D10 (μM) | D50 (μM) | D90 (μM) |
| Value for Particles | 18.619 | 56.528 | 130.950 |
| Prepared in Part I | | | |

### Example 6—Analysis of Knee Swelling Adverse Effects in Rat Knee Due to Different Formulations Containing Particles of 3-(3-Methoxy-4-((4-methoxybenzyl)oxy)benzyl)-6-(1-methyl-1H-pyrazol-4-yl)-3H-imidazo[4,5-b]pyridin-2-amine Monohydrate

Four different formulations containing particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate were prepared and evaluated for the presence of the adverse effect of knee swelling upon injection into the knee of a rat. Experimental procedures and results are provided below.

### Part I—Experimental Procedure

The test Formulations set forth in the following table were prepared.

| Formu- | |
|---|---|
| lation | |
| No. | Description of Formulation |
| 1 | This formulation contains only vehicle (which is a mixture of |
| | water, sorbitol, povidone K17, and phosphate buffer) |
| 2 | A suspension of containing vehicle and micronized particles |
| | of 3-(3-methoxy-4-((4-methoxybenzyl)oxy)benzyl)-6-(1-methyl- |
| | 1H-pyrazol-4-yl)-3H-imidazo[4,5-b]pyridin-2-amine |
| | monohydrate Form 1. The micronized particles had a D90 less |
| | than 20 μm. |
| 3 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 28 μm. |
| 4 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 56 μm. |
| 5 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 182 μm. |

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 were prepared according to procedures set forth in international patent application WO 2016/100677, and then the resulting particles were subjected to micronization to produce the micronized particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 having a D90 less than 20 μm.

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 having the size set forth in the table above for Formulations 3 and 4 were prepared by wet milling larger particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3.

Test Formulations 2 to 5 were prepared by admixing vehicle with the specified particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate, to generate a suspension formulation.

In the rat experiment, each test formulation was intra-articularly injected to the knee of three SD rats. The size of the rat knee was measured for three days. Rat knee swelling is identified by an increase in the size of the rat knee.

### Part II—Results

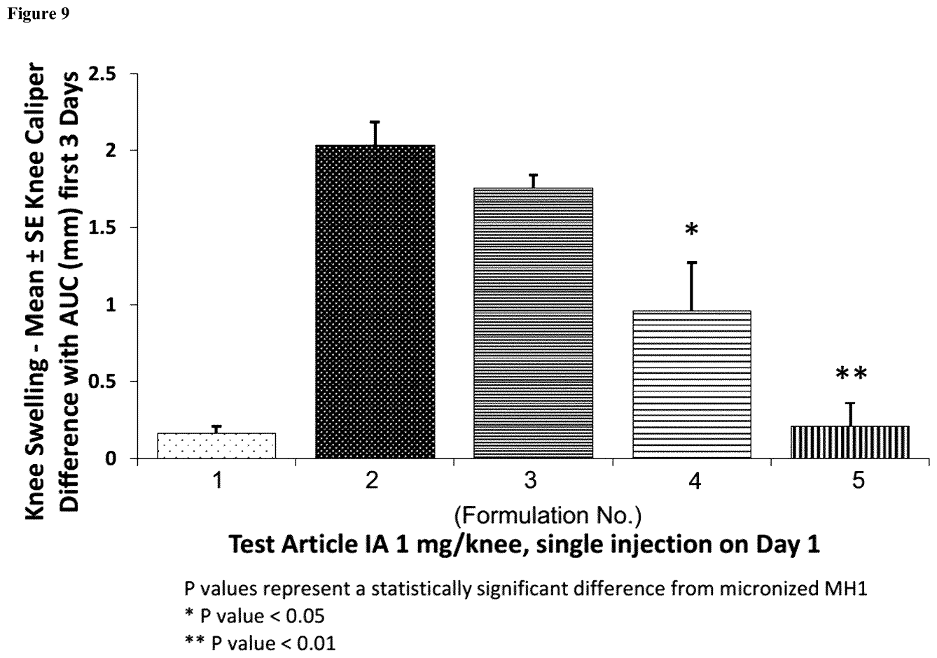

Results are shown in FIG. 9, which provide the rat knee caliper difference with AUC on day three compared to start of the experiment (i.e., day 0). Results in FIG. 9 show that while Formulations 2 and 3 result in significant knee swelling in the rats, Formulation 4 and 5 resulted in much less knee swelling in the rats. These results in FIG. 9 demonstrate that the larger particle size of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 used in Formulations 4 and 5 results in substantially less of the adverse event of knee swelling in the rat compared to the smaller particle size of 3-(3-methoxy-4-((4-methoxybenzyl)oxy)benzyl)-6-(1-methyl-1H-pyrazol-4-yl)-3H-imidazo[4,5-b]pyridin-2-amine monohydrate used in Formulations 2 and 3.

### Example 7—Additional Analysis of Knee Swelling Adverse Effects in Rat Knee Due to Different Formulations Containing Particles of 3-(3-Methoxy-4-((4-methoxybenzyl)oxy) benzyl)-6-(1-methyl-1H-pyrazol-4-yl)-3H-imidazo[4,5-b]pyridin-2-amine Monohydrate

Five different formulations containing particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate were prepared and evaluated for the presence of the adverse effect of knee swelling upon injection into the knee of a rat. Experimental procedures and results are provided below.

### Part I—Experimental Procedure

The test Formulations set forth in the following table were prepared.

| Formu- | |
|---|---|
| lation | |
| No. | Description of Formulation |
| 1 | This formulation contains only vehicle (which is a mixture of |
| | water, sorbitol, povidone K17, and phosphate buffer) |
| 2 | A suspension of containing vehicle and micronized particles |
| | of 3-(3-methoxy-4-((4-methoxybenzyl)oxy)benzyl)-6-(1-methyl- |
| | 1H-pyrazol-4-yl)-3H-imidazo[4,5-b]pyridin-2-amine |
| | monohydrate Form 1. The micronized particles had a D90 less |
| | than 20 μm. |
| 3 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 56 μm. |
| 4 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of about 90 μm. |
| 5 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of about 122 μm. |
| 6 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 182 μm. |

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 were prepared according to procedures set forth in international patent application WO 2016/100677, and then the resulting particles were subjected to micronization to produce the micronized particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 having a D90 less than 20 μm.

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 having the size set forth in the table above for Formulations 3-5 were prepared by wet milling larger particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3.

Test Formulations 2 to 6 were prepared by admixing vehicle with the specified particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate, to generate a suspension formulation.

In the rat experiment, each test formulation was intra-articularly injected to the knee of six SD rats (which comprised three male SD rats and three female SD rats). The size of the rat knee was measured for three days. Rat knee swelling is identified by an increase in the size of the rat knee.

### Part II—Results

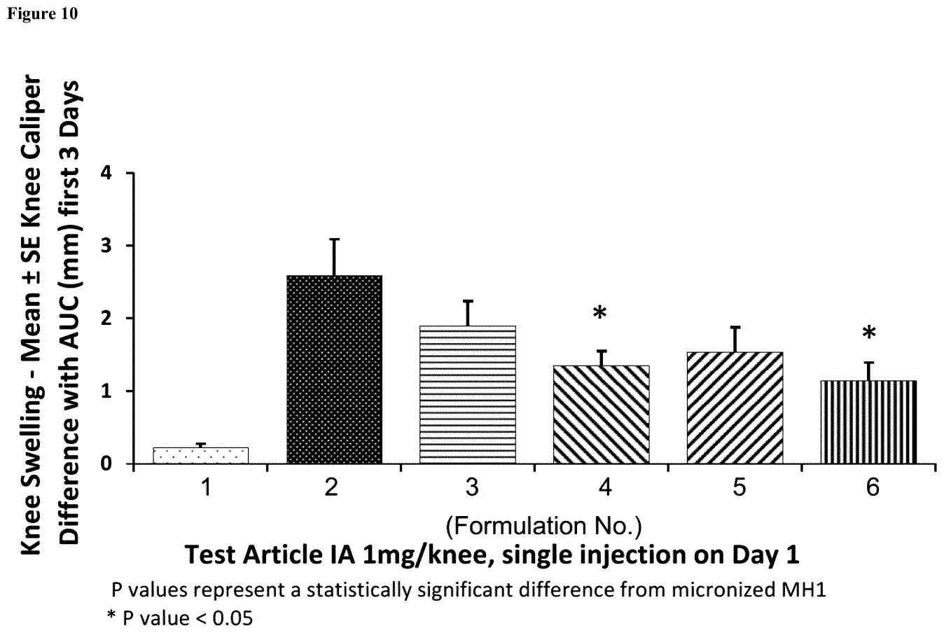

Results are shown in FIG. 10, which provide the rat knee caliper difference with AUC on day three compared to start of the experiment (i.e., day 0). Results in FIG. 10 show that while Formulation 2 resulted in significant knee swelling in the rats, Formulations 3 to 6 resulted in much less knee swelling in the rats. These results in FIG. 10 demonstrate that the larger particle size of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 used in Formulations 3 to 6 results in substantially less of the adverse event of knee swelling in the rat compared to the smaller particle size of 3-(3-methoxy-4-((4-methoxybenzyl)oxy)benzyl)-6-(1-methyl-1H-pyrazol-4-yl)-3H-imidazo[4,5-b]pyridin-2-amine monohydrate used in Formulation 2.

### Example 8—Analysis of Efficacy in Treating Rat Knee Pain with Different Formulations Containing Particles of 3-(3-Methoxy-4-((4-methoxybenzyl)oxy)benzyl)-6-(1-methyl-1H-pyrazol-4-yl)-3H-imidazo[4,5-b]pyridin-2-amine Monohydrate

Four different formulations containing particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate were prepared and evaluated for ability treat pain in an animal model of osteoarthritis pain. Experimental procedures and results are provided below.

### Part I—Experimental Procedure

The test Formulations set forth in the following table were prepared.

| Formu- | |
|---|---|
| lation | |
| No. | Description of Formulation |
| 1 | This formulation contains only vehicle (which is a mixture of |
| | water, sorbitol, povidone K17, and phosphate buffer) |
| 2 | A suspension of containing triamcinolone acetonide (TCA) and |
| | vehicle |
| 3 | A suspension of containing vehicle and micronized particles |
| | of 3-(3-methoxy-4-((4-methoxybenzyl)oxy)benzyl)-6-(1-methyl- |
| | 1H-pyrazol-4-yl)-3H-imidazo[4,5-b]pyridin-2-amine |
| | monohydrate Form 1. The micronized particles had a D90 less |
| | than 20 μm. |
| 4 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 28 μm. |
| 5 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 56 μm. |
| 6 | A suspension of containing vehicle and particles of 3-(3- |
| | methoxy-4-((4-methoxybenzyl)oxy)benzyl)-6-(1-methyl-1H- |
| | pyrazol-4-yl)-3H-imidazo[4,5-b]pyridin-2-amine monohydrate |
| | Form 3, where the particles have a D90 of 182 μm. |

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 were prepared according to procedures set forth in international patent application WO 2016/100677, and then the resulting particles were subjected to micronization to produce the micronized particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 1 having a D90 less than 20 μm.

Particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 having the size set forth in the table above for Formulations 4 and 5 were prepared by wet milling larger particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3.

Test Formulation 2 was prepared by admixing vehicle with TCA, to generate a suspension formulation. Test Formulations 3 to 6 were prepared by admixing vehicle with the specified particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate, to generate a suspension formulation.

In the rat experiment, Lewis rats were primed by injecting PGPS antigen into the knee of the rate; this was deemed day −21 of the experiment. Test formulations were administered to the rat knee by injection on day −7 of the experiment. Test formulation 2 was administered to the rat knee in an amount sufficient to provide a 1 mg dose of TCA. Test formulations 3 through 6 were administered to the rat knee in an amount sufficient to provide a 1 mg dose of 3-(3-methoxy-4-((4-methoxybenzyl)oxy)benzyl)-6-(1-methyl-1H-pyrazol-4-yl)-3H-imidazo[4,5-b]pyridin-2-amine monohydrate. A corresponding volume of Formulation 1 was administered to a rat control group. Antigen was administered to the rats by intravenous injection on day 0 and day 14 of the experiment. The antigen leads to a marked inflammatory reaction in the initially injected knee, manifest by pain behavior and knee swelling. Ability of the test formulation to prevent the inflammatory reaction is evidence of beneficial effects of the test formulation. For each of Formulations 1 to 6, the Formulation was administered to 10 male Lewis rats.

### Part II—Results

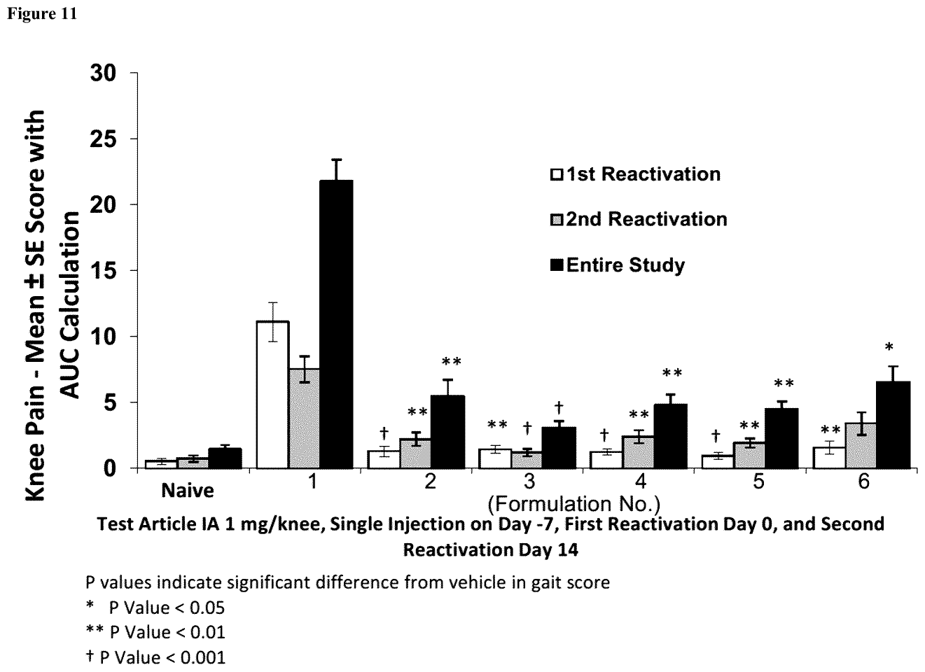

Results are shown in FIG. 11 which provides a gait score indicating the extent of knee pain experienced by the rat. Results in FIG. 11 show that each of Formulations 2 to 6 reduce knee pain in this animal model of osteoarthritis relative to vehicle control (i.e., Formulation 1).

### Example 9—Analysis of Inflammatory Adverse Effects in Rat Paw for Different Formulations Containing 3-(3-Methoxy-4-((4-methoxybenzyl)oxy)benzyl)-6-(1-methyl-1H-pyrazol-4-yl)-3H-imidazo[4,5-b]pyridin-2-amine Monohydrate

Multiple formulations containing 3-(3-methoxy-4-((4-methoxybenzyl)oxy)benzyl)-6-(1-methyl-1H-pyrazol-4-yl)-3H-imidazo[4,5-b]pyridin-2-amine monohydrate were prepared and evaluated for the presence of the adverse effect of inflammation upon injection of the formulation into the paw of a rat. Experimental procedures and results are provided below.

### Part I—Experimental Procedure

The test Formulations set forth in the following table were prepared.

| Formulation No. | Description of Formulation |
|---|---|
| 1 | Vehicle for Aqueous Suspension (which is a mixture of water, sorbitol, |
| | povidone K17, and phosphate buffer) |
| 2 | A suspension of containing aqueous vehicle from Formulation 1 and |
| | particles of 3-(3-methoxy-4-((4-methoxybenzyl)oxy)benzyl)-6-(1-methyl- |
| | 1H-pyrazol-4-yl)-3H-imidazo[4,5-b]pyridin-2-amine monohydrate Form |
| | 3 having a D90 particle size of 13 μm |
| 3 | A suspension of containing aqueous vehicle from Formulation 1 and |
| | particles of 3-(3-methoxy-4-((4-methoxybenzyl)oxy)benzyl)-6-(1-methyl- |
| | 1H-pyrazol-4-yl)-3H-imidazo[4,5-b]pyridin-2-amine monohydrate Form |
| | 3 having a D90 particle size of 130 μm |
| 4 | Lipid nanoparticle formulation having small size particles of 3-(3- |
| | methoxy-4-((4-methoxybenzyl)oxy)benzyl)-6-(1-methyl-1H-pyrazol-4- |
| | yl)-3H-imidazo[4,5-b]pyridin-2-amine monohydrate |
| 5 | Lipid nanoparticle formulation having medium size particles of 3-(3- |
| | methoxy-4-((4-methoxybenzyl)oxy)benzyl)-6-(1-methyl-1H-pyrazol-4- |
| | yl)-3H-imidazo[4,5-b]pyridin-2-amine monohydrate |
| 6 | Lipid nanoparticle formulation having large size particles of 3-(3- |
| | methoxy-4-((4-methoxybenzyl)oxy)benzyl)-6-(1-methyl-1H-pyrazol-4- |
| | yl)-3H-imidazo[4,5-b]pyridin-2-amine monohydrate |
| 7 | First emulsion formulation containing fully dissolved 3-(3-methoxy-4-((4- |
| | methoxybenzyl)oxy)benzyl)-6-(1-methyl-1H-pyrazol-4-yl)-3H- |
| | imidazo[4,5-b]pyridin-2-amine monohydrate |
| 8 | Second emulsion formulation containing fully dissolved 3-(3-methoxy-4- |
| | ((4-methoxybenzyl)oxy)benzyl)-6-(1-methyl-1H-pyrazol-4-yl)-3H- |
| | imidazo[4,5-b]pyridin-2-amine monohydrate |
| 9 | Third emulsion formulation containing fully dissolved 3-(3-methoxy-4- |
| | ((4-methoxybenzyl)oxy)benzyl)-6-(1-methyl-1H-pyrazol-4-yl)-3H- |
| | imidazo[4,5-b]pyridin-2-amine monohydrate |
| 10 | Fourth emulsion formulation containing fully dissolved 3-(3-methoxy-4- |
| | ((4-methoxybenzyl)oxy)benzyl)-6-(1-methyl-1H-pyrazol-4-yl)-3H- |
| | imidazo[4,5-b]pyridin-2-amine monohydrate |
| 11 | Fifth emulsion formulation containing fully dissolved 3-(3-methoxy-4- |
| | ((4-methoxybenzyl)oxy)benzyl)-6-(1-methyl-1H-pyrazol-4-yl)-3H- |
| | imidazo[4,5-b]pyridin-2-amine monohydrate |
| 12 | Sixth emulsion formulation containing fully dissolved 3-(3-methoxy-4- |
| | ((4-methoxybenzyl)oxy)benzyl)-6-(1-methyl-1H-pyrazol-4-yl)-3H- |
| | imidazo[4,5-b]pyridin-2-amine monohydrate |
| 13 | Emulsion formulation containing just vehicle (same vehicle used in |
| | Formulations 7 to 12) |
| 14 | Mixture containing aqueous vehicle from Formulation 1 and micronized |
| | particles of 3-(3-methoxy-4-((4-methoxybenzyl)oxy)benzyl)-6-(1-methyl- |
| | 1H-pyrazol-4-yl)-3H-imidazo[4,5-b]pyridin-2-amine monohydrate Form |
| | 1 having a D90 less than 20 μm. |

An aliquot of Test Formulation was injected into the paw of a rat, and the paw was monitored for inflammation for a period of 96 hours. Each Test Formulation was administered to three different rats. The extent of inflammation was evaluated as a combination assessment of the visual appearance of inflammation, increased paw thickness determined with a caliper, and tenderness to palpation.

### Part II—Results

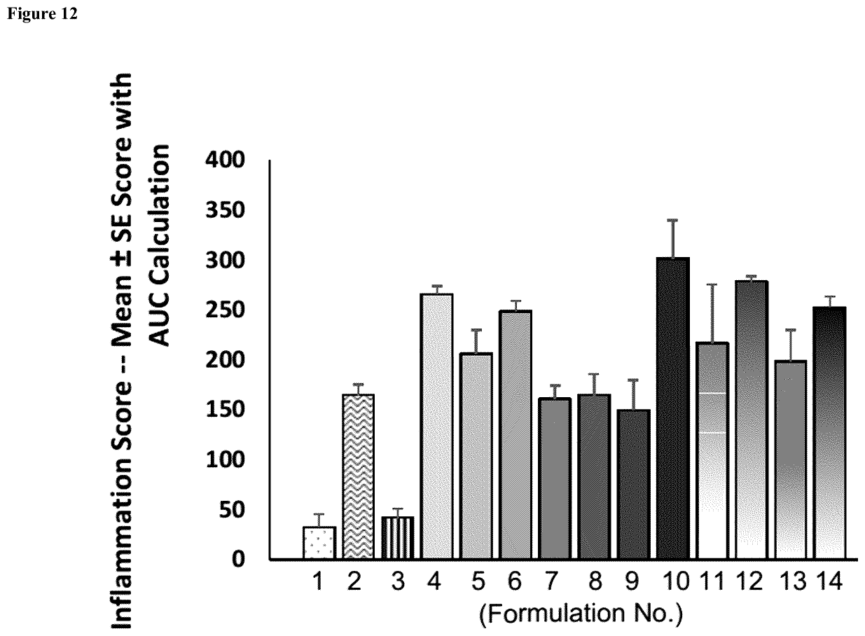

Results are shown in FIG. 12 which provides an inflammation score indicating the extent of rat paw inflammation observed following injection of test formulation. Results in FIG. 12 show that Formulation 2 and each of Formulations 4 to 14 caused substantial inflammation in the rat paw. Formulation 3 was much better tolerated by the rat paw, resulting in very little inflammation in the rat paw.

### Example 10—Preparation of Particles Containing 3-(3-Methoxy-4-((4-methoxybenzyl)oxy)benzyl)-6-(1-methyl-1H-pyrazol-4-yl)-3H-imidazo[4,5-b]pyridin-2-amine Monohydrate Form 3 by Wet Milling

A solution containing 3-(3-methoxy-4-((4-methoxybenzyl)oxy)benzyl)-6-(1-methyl-1H-pyrazol-4-yl)-3H-imidazo[4,5-b]pyridin-2-amine monohydrate (9 g) and 62.5 volumes acetone/water (5/1, v/v) was stirred at 55° C. The solution was then cooled to 50° C., and then charged with 2.0% of particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3. Then, the resulting mixture was aged at 50° C. for 3 hour, and then cooled to 45° C. over 0.8 hours (cooling rate: 0.1° C./min). Next, the resulting mixture was stirred at 45° C. for 1 h, and then the mixture was cooled to 40° C. over 0.8 hours (cooling rate: 0.1° C./min).

Then, the resulting mixture was stirred at 40° C. for 1 h, and then the mixture was cooled to 5° C. over 5.8 hours (cooling rate: 0.1° C./min). Next, the resulting mixture was stirred at 5° C. for 2 hours.

Next, the mixture was heated to 50° C. over 2 hours, then the resulting mixture was stirred at 50° C. for 2 hours, and then the mixture was cooled to 45° C. over 0.8 hours (cooling rate: 0.1° C./min). Next the resulting mixture was stirred at 45° C. for 1 h, and then the mixture was cooled to 40° C. over 0.8 hours (cooling rate: 0.1° C./min). Next, the resulting mixture was stirred at 40° C. for 1 h. Thereafter, the resulting mixture was cooled to 5° C. over 5.8 hours (cooling rate: 0.1° C./min), and then the mixture was stirred at 5° C. for 2 hours.

Next, the mixture was heated to 50° C. over 2 hours, then the resulting mixture was stirred at 50° C. for 2 hours, and then the mixture was cooled to 45° C. over 0.8 hours (cooling rate: 0.1° C./min). Next the resulting mixture was stirred at 45° C. for 1 h, and then the mixture was cooled to 40° C. over 0.8 hours (cooling rate: 0.1° C./min). Next, the resulting mixture was stirred at 40° C. for 1 h. Thereafter, the resulting mixture was cooled to 5° C. over 5.8 hours (cooling rate: 0.1° C./min), and then the mixture was stirred at 5° C. for 2 hours.

Next, the mixture was heated to 50° C. over 2 hours, then the resulting mixture was stirred at 50° C. for 2 hours, and then the mixture was cooled to 45° C. over 0.8 hours (cooling rate: 0.1° C./min). Next the resulting mixture was stirred at 45° C. for 1 h, and then the mixture was cooled to 40° C. over 0.8 hours (cooling rate: 0.1° C./min). Next, the resulting mixture was stirred at 40° C. for 1 h. Thereafter, the resulting mixture was cooled to 5° C. over 5.8 hours (cooling rate: 0.1° C./min), and then the mixture was stirred at 5° C. for 2 hours.

Next, the mixture was heated to 50° C. over 2 hours, then the resulting mixture was stirred at 50° C. for 2 hours, and then the mixture was cooled to 45° C. over 0.8 hours (cooling rate: 0.1° C./min). Next the resulting mixture was stirred at 45° C. for 1 h, and then the mixture was cooled to 40° C. over 0.8 hours (cooling rate: 0.1° C./min). Next, the resulting mixture was stirred at 40° C. for 1 h. Thereafter, the resulting mixture was cooled to 5° C. over 5.8 hours (cooling rate: 0.1° C./min), and then the mixture was stirred at 5° C. for 2 hours.

Thereafter, the resulting mixture was stirred at 5° C. for 10 hours.

The resulting solid particles (having a D90 of 283 μm) were wet milled using 2P/4M with tip speed 13 m/s for 40 cycles to produce particles having a D90 of 127 μm.

An aliquot of particles having a D90 of 127 μm were subjected to further wet milling using a 2P/4M with tip speed 18 m/s for 40 cycles to particles having a D90 of 89.7 μm. The resulting particles were subjected to filtration, and the wet cake was washed with two volumes of pre-cooled acetone/water (5/1, v/v) two times. The resulting wet cake was dried at 25° C. for 6 h under vacuum to provide particles of 3-(3-methoxy-4-((4-methoxybenzyl)oxy)benzyl)-6-(1-methyl-1H-pyrazol-4-yl)-3H-imidazo[4,5-b]pyridin-2-amine monohydrate Form 3 having a D90 of 90.6 μm.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. Particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D50 particle size in the range of from 40 μm to 75 μm.

2. (canceled)

3. (canceled)

4. (canceled)

5. The particles of claim 1, wherein the particles have a D50 particle size in the range of from 55 μm to 59 μm.

6. The particles of claim 1, wherein the particles have a D50 particle size in the range of from 56 μm to 57 μm.

7. (canceled)

8. (canceled)

9. (canceled)

10. The particles of claim 1, wherein the particles have a D90 particle size in the range of from 121 μm to 141 μm.

11. The particles of claim 1, wherein the particles have a D90 particle size in the range of from 126 μm to 136 μm.

12. The particles of claim 6, wherein the particles have a D90 particle size in the range of from 129 μm to 133 μm.

13. The particles of claim 6, wherein the particles have a D90 particle size in the range of from 130 μm to 132 μm.

14. (canceled)

15. (canceled)

16. (canceled)

17. (canceled)

18. The particles of claim 1, wherein the particles have a D10 particle size in the range of from 16 μm to 22 μm.

19. (canceled)

20. The particles of claim 13, wherein the particles have a D10 particle size in the range of from 18 μm to 19 μm.

21. (canceled)

22. (canceled)

23. Particles of the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate having a D90 particle size in the range of from 70 μm to 150 μm.

24. The particles of claim 23, wherein the particles have a D50 particle size in the range of from 35 μm to 45 μm.

25. (canceled)

26. (canceled)

27. The particles of claim 23, wherein the particles have a D90 particle size in the range of from 80 μm to 100 μm.

28. The particles of claim 23, wherein the particles have a D90 particle size in the range of from 85 μm to 95 μm.

29. The particles of claim 23, wherein the particles have a D90 particle size in the range of from 88 μm to 92 μm.

30. The particles of claim 23, wherein the particles have a D90 particle size of about 90 μm.

31. The particles of claim 23, wherein the particles have a D90 particle size in the range of from 110 μm to 130 μm.

32. The particles of claim 23, wherein the particles have a D90 particle size in the range of from 120 μm to 124 μm.

33. The particles of claim 23, wherein the particles have a D90 particle size of about 120 μm.

34. (canceled)

35. The particles of claim 23, wherein the particles have a D10 particle size in the range of from 20 μm to 30 μm.

36. The particles of claim 23, wherein the compound 3-(3-methoxy-4-((4-methoxybenzyl)oxy)benzyl)-6-(1-methyl-1H-pyrazol-4-yl)-3H-imidazo[4,5-b]pyridin-2-amine monohydrate is in crystalline form.

37. (canceled)

38. (canceled)

39. (canceled)

40. The particles of claim 36, wherein at least 90% by weight of the compound in the particles is in the form of plate-like crystals.

41. (canceled)

42. (canceled)

43. The particles of claim 36, wherein less than 5% by weight of the compound in the particles is in the form of needle-like crystals.

44. The particles of claim 23, wherein the compound is 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form exhibiting an X-ray powder diffraction pattern comprising peaks at the following diffraction angles (2θ): 14.9±0.2, 20.2 ±0.2, 20.7±0.2, 21.4±0.2, 25.1±0.2, 28.0±0.2, and 30.0±0.2.

45. (canceled)

46. (canceled)

47. (canceled)

48. The particles of claim 44, wherein the X-ray powder diffraction pattern further comprises peaks at the following diffraction angles (2θ): 12.8±0.2, 13.2±0.2, 17.3±0.2, 19.0±0.2, 23.9±0.2, 26.5±0.2, 28.4±0.2, and 28.8±0.2.

49. (canceled)

50. (canceled)

51. (canceled)

52. The particles of claim 44, wherein the compound is characterized by the following X-ray powder diffraction pattern expressed in terms of diffraction angle 2θ, inter-planar distances d, and relative intensity (expressed as a percentage with respect to the most intense peak):

| Angle [2θ] | d-spacing [Å] | Relative Intensity [%] |
|---|---|---|
| 12.78 | 6.93 | 7.3 |
| 13.19 | 6.71 | 8.1 |
| 14.87 | 5.96 | 100.0 |
| 16.62 | 5.33 | 19.3 |
| 17.34 | 5.12 | 8.9 |
| 19.03 | 4.66 | 11.8 |
| 20.19 | 4.40 | 49.1 |
| 20.67 | 4.30 | 31.0 |
| 21.40 | 4.15 | 40.7 |
| 22.56 | 3.94 | 28.6 |
| 22.90 | 3.88 | 26.1 |
| 23.93 | 3.72 | 14.4 |
| 25.09 | 3.55 | 64.4 |
| 26.53 | 3.36 | 7.4 |
| 28.00 | 3.19 | 41.6 |
| 28.35 | 3.15 | 10.5 |
| 28.83 | 3.10 | 6.2 |
| 29.95 | 2.98 | 42.4 |

53. (canceled)

54. (canceled)

55. (canceled)

56. (canceled)

57. (canceled)

58. A pharmaceutical composition comprising the particles of claim 23 and a pharmaceutically acceptable carrier.

59. A method of treating a disease or condition selected from the group consisting of an inflammatory disease, autoimmune disease, pain, osteoarthritis, defect of bone metabolism, and cancer, comprising administering a therapeutically effective amount of the particles of claim 23 to a subject in need thereof to treat the disease or condition.

60. (canceled)

61. (canceled)

62. (canceled)

63. The method of claim 59, wherein the disease or condition is pain due to osteoarthritis.

64. (canceled)

65. The method of claim 59, wherein the disease or condition is osteoarthritis.

66. (canceled)

67. (canceled)

68. A method of inhibiting the activity of a tropomyosin-related kinase, comprising contacting a tropomyosin-related kinase with an effective amount of the particles of claim 23 to inhibit the activity of said tropomyosin-related kinase.

69. (canceled)

70. (canceled)

71. (canceled)

72. A method of inhibiting the activity of a cellular receptor for colony stimulating factor-1, comprising contacting said cellular receptor for colony stimulating factor-1 with an effective amount of the particles of claim 23 to inhibit the activity of said cellular receptor for colony stimulating factor-1.

73. A method of preparing the particles of claim 23, comprising the steps of:
a. admixing (i) a first solution having a temperature of about 50° C. and containing acetone, water, and 3-(3-methoxy-4-((4-methoxybenzyl)oxy)benzyl)-6-(1-methyl-1H-pyrazol-4-yl)-3H-imidazo[4,5-b]pyridin-2-amine and (ii) an aliquot of 3-(3-methoxy-4-((4-methoxybenzyl)oxy)benzyl)-6-(1-methyl-1H-pyrazol-4-yl)-3H-imidazo[4,5-b]pyridin-2-amine monohydrate in crystalline form to provide a first mixture;
b. maintaining the first mixture at a temperature in the range of from 42° C. to 48° C. for a duration of about 1 hour, then cooling the first mixture to a temperature in the range of from 37° C. to 42° C. and maintaining the first mixture at a temperature in the range of from 37° C. to 42° C. for about 1 hour, then cooling the first mixture to a temperature in the range of from 2° C. to 8° C. and maintaining the first mixture at a temperature in the range of from 2° C. to 8° C. for about 1 hour, to produce a second mixture;
c. heating the second mixture to a temperature in the range of from 48° C. to 53° C., maintaining the second mixture at a temperature in the range of from 48° C. to 53° C. for about 3 hours, then cooling the second mixture to a temperature in the range of from 43° C. to 47° C. and maintaining the second mixture at a temperature in the range of from 43° C. to 47° C. for about 1 hour, then cooling the second mixture to a temperature in the range of from 37° C. to 42° C. and maintaining the second mixture at a temperature in the range of from 37° C. to 42° C. for about 1 hour, then cooling the second mixture to a temperature in the range of from 2° C. to 8° C. and maintaining the second mixture at a temperature in the range of from 2° C. to 8° C. for about 1 hour, to produce a third mixture;
d. heating the third mixture to a temperature in the range of from 48° C. to 53° C., maintaining the third mixture at a temperature in the range of from 48° C. to 53° C. for about 3 hours, then cooling the third mixture to a temperature in the range of from 43° C. to 47° C. and maintaining the third mixture at a temperature in the range of from 43° C. to 47° C. for about 1 hour, then cooling the third mixture to a temperature in the range of from 37° C. to 42° C. and maintaining the third mixture at a temperature in the range of from 37° C. to 42° C. for about 1 hour, then cooling the third mixture to a temperature in the range of from 2° C. to 8° C. and maintaining the third mixture at a temperature in the range of from 2° C. to 8° C. for about 1 hour, to produce a crystallization mixture; and
e. isolating said particles of claim 1 from the crystallization mixture.

74-82. (canceled)
